SEQUENCE LISTING (10 sequences)

SEQ ID NO: 1 - protein, 5 aa, synthetic construct
SYSMN

SEQ ID NO: 2 - protein, 17 aa, synthetic construct
SISSSSSYIYYAESVKG

SEQ ID NO: 3 - protein, 9 aa, synthetic construct
VGLFGPFDI

SEQ ID NO: 4 - protein, 11 aa, synthetic construct
RASQSVSRYLG

SEQ ID NO: 5 - protein, 7 aa, synthetic construct
GASTRAT

SEQ ID NO: 6 - protein, 9 aa, synthetic construct
QQYGSSPWT

SEQ ID NO: 7 - protein, 118 aa, synthetic construct
EVQLVESGGGLVKPGGSLRLSCAASGFTFSSYSMNWVRQAPGKGLEWVSSISSSSSYIYYAESVKGRFTISRDNAKNSLYLQMNSLRAEDTAVYYCARVGLFGPFDIWGQGTLVTVSS

SEQ ID NO: 8 - protein, 107 aa, synthetic construct
EIVLTQSPGTLSLSPGERATLSCRASQSVSRYLGWYQQKPGQAPRLLIYGASTRATGIPDRFSGSGSGTDFTLTITRLEPEDFAVYYCQQYGSSPWTFGQGTKVEIK

SEQ ID NO: 9 - protein, 447 aa, synthetic construct
EVQLVESGGGLVKPGGSLRLSCAASGFTFSSYSMNWVRQAPGKGLEWVSSISSSSSYIYYAESVKGRFTISRDNAKNSLYLQMNSLRAEDTAVYYCARVGLFGPFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 214 aa, synthetic construct
EIVLTQSPGTLSLSPGERATLSCRASQSVSRYLGWYQQKPGQAPRLLIYGASTRATGIPDRFSGSGSGTDFTLTITRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0183389 A1
(43) Pub. Date: Jul. 2, 2026

(54) METHODS OF TREATING PANCREATIC CANCER USING AN ANTI-CTLA4 ANTIBODY

(71) Applicant: AGENUS INC., Lexington, MA (US)

(72) Inventors: Dhan Sidhartha CHAND, Lexington, MA (US); Joseph GROSSMAN, Lexington, MA (US)

(73) Assignee: AGENUS INC., Lexington, MA (US)

(21) Appl. No.: 19/129,651

(22) PCT Filed: Nov. 15, 2023
(86) PCT No.: PCT/US2023/079858

§ 371 Date: May 13, 2025

Related U.S. Application Data
(60) Provisional application No. 63/383,724, filed on Nov. 15, 2022.

(51) Int. Cl. A61K 39/395; A61K 31/337; A61K 31/7068; A61K 39/00; A61P 35/00; C07K 16/28

(52) U.S. Cl. CPC A61K 39/3955; A61K 31/337; A61K 31/7068; A61P 35/00; C07K 16/2818; A61K 2039/505; A61K 2039/54; A61K 2039/545; C07K 2317/72

(57) ABSTRACT

Provided are methods for treating pancreatic cancer with an antibody that specifically binds to human Cytotoxic T-Lymphocyte Antigen 4 (CTLA-4).

### RELATED APPLICATIONS

This application claims priority to U.S. Provisional Patent Application Ser. No. 63/383,724, filed Nov. 15, 2022, the entire disclosure of which is hereby incorporated by reference herein.

### REFERENCE TO SEQUENCE LISTING

This application contains a sequence listing which has been submitted electronically in ST.26 format and is hereby incorporated by reference in its entirety (said ST.26 copy, created on Nov. 14, 2023, is named “205365_seqlist.xml” and is 10,360 bytes in size).

### BACKGROUND

Although tenth in terms of incidence, pancreatic carcinoma it is the third leading cause of cancer deaths in the United States. This high mortality-to-incidence ratio is due to patients commonly being diagnosed at an advanced stage. Thus, in addition to advancing early detection in pancreatic cancer patients to reduce overall mortality, new therapies for advanced disease are urgently needed.

Currently, the standard initial treatment for patients with metastatic pancreatic cancer is systemic combination chemotherapy (e.g., 5-fluorouracil, oxaliplatin, irinotecan, nab-paclitaxel, gemcitabine). However, these systemic therapies have only demonstrated modest efficacy and lead to a median overall survival of less than one year in patients with metastatic pancreatic cancer. Also, to date, immune checkpoint inhibitors have not shown much success in pancreatic cancer, except in those that with high microsatellite instability (MSI-H), which is only about 1% of pancreatic cancers.

Accordingly, there remains a need for novel and efficacious methods of treating metastatic pancreatic cancer.

### SUMMARY

The instant disclosure is directed to methods for treating pancreatic cancer with an antibody that specifically binds to human Cytotoxic T-Lymphocyte Antigen 4 (CTLA-4), alone or in combination with nab-paclitaxel and/or gemcitabine. Also provided herein are particular methods for administering an antibody that specifically binds to human CTLA-4 that results in a reduction of tumor burden in a subject. In an embodiment, the method comprises a therapeutically effective amount that safely and effectively treats metastatic pancreatic cancer.

In an aspect, provided herein is a method of treating pancreatic cancer in a subject in need thereof, the method comprising administering to the subject an antibody that specifically binds to human Cytotoxic T-Lymphocyte Antigen 4 (CTLA-4) at a dose of 25 mg to 250 mg, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an aspect, provided herein is a method of enhancing the activation of T cells in a subject who has pancreatic cancer, the method comprising administering to the subject an antibody that specifically binds to human CTLA-4 at a dose of 25 mg to 250 mg, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In certain embodiments the antibody is administered at a dose of about 25 mg to 200 mg. In an embodiment, the antibody is administered at a dose of 25 mg to 150 mg. In an embodiment, the antibody is administered at a dose of 50 mg to 150 mg. In an embodiment, the antibody is administered at a dose of 50 mg to 200 mg. In an embodiment, the antibody is administered at a dose of 25 mg, 50 mg, 75 mg, 100 mg, 125 mg, 150 mg, 175 mg, 200 mg, 225 mg, or 250 mg.

In an embodiment, the antibody is administered intravenously. In an embodiment, the antibody is administered by intravenous infusion over about 30 minutes.

In an embodiment, the antibody is administered once weekly. In an embodiment, the antibody is administered once every 2 weeks. In an embodiment, the antibody is administered once every 3 weeks. In an embodiment, the antibody is administered once every 4 weeks. In an embodiment, the antibody is administered once every 5 weeks. In an embodiment, the antibody is administered once every 6 weeks.

In an embodiment, the antibody is administered intravenously at a dose of 25 mg once every 6 weeks. In an embodiment, the antibody is administered intravenously at a dose of 50 mg once every 6 weeks. In an embodiment, the antibody is administered intravenously at a dose of 75 mg once every 6 weeks. In an embodiment, the antibody is administered intravenously at a dose of 100 mg once every 6 weeks. In an embodiment, the antibody is administered intravenously at a dose of 125 mg once every 6 weeks. In an embodiment, the antibody is administered intravenously at a dose of 150 mg once every 6 weeks. In an embodiment, the antibody is administered intravenously at a dose of 175 mg once every 6 weeks. In an embodiment, the antibody is administered intravenously at a dose of 200 mg once every 6 weeks. In an embodiment, the antibody is administered intravenously at a dose of 225 mg once every 6 weeks. In an embodiment, the antibody is administered intravenously at a dose of 250 mg once every 6 weeks.

In an embodiment, the dose is a therapeutically effective amount.

In an embodiment, the pancreatic cancer is pancreatic ductal adenocarcinoma. In an embodiment, the pancreatic cancer is unresectable. In an embodiment, the pancreatic cancer is metastatic. In an embodiment, the pancreatic cancer is relapsed and/or refractory.

In an embodiment, the subject has received at least one prior chemotherapy. In an embodiment, the at least one prior chemotherapy is 5-fluorouracil, leucovorin, irinotecan, or oxaliplatin. In an embodiment, the subject has previously been treated with 5-fluorouracil, leucovorin, irinotecan, and oxaliplatin. In an embodiment, the pancreatic cancer relapsed following a standard of care treatment.

In an embodiment, the method further comprises administering nab-paclitaxel to the subject. In an embodiment, nab-paclitaxel is administered at a dose of 75 mg/m², 100 mg/m², or 125 mg/m². In an embodiment, nab-paclitaxel is administered once every week. In an embodiment, nab-paclitaxel is administered on days 1, 8, and 15 of a 6 week cycle.

In an embodiment, the method further comprises administering gemcitabine to the subject. In an embodiment, gemcitabine is administered at a dose of 600 mg/m², 800 mg/m², or 1000 mg/m². In an embodiment, gemcitabine is administered once every week. In an embodiment, gemcitabine is administered on days 1, 8, and 15 of a 6 week cycle

In an embodiment, the antibody is administered on day 1 of a 6 week cycle and nab-paclitaxel and gemcitabine are administered on days 1, 8, and 15 of the 6 week cycle. In an embodiment, the antibody is administered to the subject before nab-paclitaxel and gemcitabine. In an embodiment, nab-paclitaxel is administered before gemcitabine.

In an embodiment, the cancer is refractory to a standard of care treatment. In an embodiment, the standard of care treatment is chemotherapy or radiation. In an embodiment, the standard of care treatment is 5-fluorouracil, leucovorin, irinotecan, and/or oxaliplatin.

In an embodiment, the method reduces tumor size in the subject. In an embodiment, the method increases T-cell activation in the subject. In an embodiment, the method reduces the level of CA 19-9, CA 125, or CEA in the subject.

In an embodiment, before administration of the antibody the subject has measurable disease on baseline imaging per RECIST 1.1. In an embodiment, before administration of the antibody the subject has an Eastern Cooperative Oncology Group performance status (PS) 0-1. In an embodiment, before administration of the antibody the subject has a predicted life expectancy of ≥12 weeks.

In an embodiment, before administration of the antibody the subject has adequate organ function as defined by one or more of: a) neutrophils ≥1500/μL; b) platelets ≥100×10³/μL; c) hemoglobin ≥9.0 g/dL; d) creatinine clearance ≥30 mL/min as measured or calculated per local institutional standards; e) Aspartate aminotransferase (AST)/alanine aminotransferase (ALT)<3.0×upper limit of normal (ULN); f) direct bilirubin <1.5×ULN (except patients with Gilbert syndrome who must have a total bilirubin level of <3.0×ULN); and/or g) serum albumin≥3.0 g/dL.

In an embodiment, the subject does not have partial or complete bowel obstruction within the last 3 months, signs/symptoms of bowel obstruction, or known radiologic evidence of impending obstruction.

In an embodiment, the subject has liver metastases, i.e., the cancer has metastasized to the liver. In an embodiment, the subject has one or more liver lesions. In an embodiment, the methods of the invention, reduce one or more liver lesions. For instance, in an embodiment, administration of the antibody that specifically binds to human Cytotoxic T-Lymphocyte Antigen 4 (CTLA-4) in combination with nab-paclitaxel and/or gemcitabine reduces liver lesions in the subject by at least about 5%, 10%, 15%, 20%, 25%, 30%, 35%, 40%, or 45% or more.

Accordingly, provided herein is a method of treating a subject with cancer that has metastasized to the liver, the method comprising administering to the subject gemcitabine, nab-paclitaxel, and an antibody that specifically binds to CTLA-4.

In an embodiment, the subject has not received an immune checkpoint inhibitor therapy prior to administration of the antibody. In an embodiment, the subject has not received more than one chemotherapy regimen prior to administration of the antibody. In an embodiment, the subject does not have a history of central nervous system (CNS) metastases. In an embodiment, the subject does not have a concurrent malignancy that requires treatment or a history of prior malignancy that was active within 2 years prior to administration of the antibody. In an embodiment, the subject has not received a cytotoxic therapy or targeted therapy, within 3 weeks prior to administration of the antibody. In an embodiment, the subject has not received other monoclonal antibody therapy, antibody-drug conjugate therapy, or radioimmunoconjugate therapy, within 4 weeks prior to administration of the antibody. In an embodiment, the subject has not received small molecule tyrosine kinase inhibitor therapy within 2 weeks prior to administration of the antibody.

In an embodiment, the subject does not have refractory ascites defined as requiring 2 or more therapeutic paracenteses within the last 4 weeks or ≥4 times within the last 90 days or ≥1 time within the last 2 weeks prior to administration of the antibody. In an embodiment, the subject does not have clinically significant cardiovascular disease.

In an embodiment, the cancer has metastasized to the liver.

In an embodiment, the antibody comprises the CDRH1, CDRH2, CDRH3, CDRL1, CDRL2, and CDRL3 amino acid sequences set forth in SEQ ID NOs: 1, 2, 3, 4, 5, and 6, respectively. In an embodiment, the antibody comprises a VH comprising the amino acid sequence set forth in SEQ ID NO: 7 and a VL comprising the amino acid sequence set forth in SEQ ID NO: 8. In an embodiment, the antibody comprises a human IgG1 heavy chain constant region comprising S239D/A330L/1332E mutations, numbered according to the EU numbering system. In an embodiment, the antibody comprises a heavy chain comprising the amino acid sequence set forth in SEQ ID NO: 9 and a light chain comprising the amino acid sequence set forth in SEQ ID NO: 10.

In an embodiment, the antibody is botensilimab.

In an aspect, provided herein is an antibody that specifically binds to human CTLA-4 for use in the treatment of pancreatic cancer, wherein the treatment is performed according to any one of the methods disclosed herein.

In an aspect, provided herein is an antibody that specifically binds to human CTLA-4 for use in the manufacture of a medicament for the treatment of pancreatic cancer, wherein the treatment is performed according to any one of the methods disclosed herein.

In an aspect, provided herein is a use of an antibody that specifically binds to human CTLA-4 for the treatment of pancreatic cancer, wherein the treatment is performed according to any one of the methods disclosed herein.

In an aspect, provided herein is an antibody that specifically binds to human CTLA-4, gemcitabine, and nab-paclitaxel for use in the treatment of pancreatic cancer, wherein the treatment is performed according to any one of the methods disclosed herein.

In an aspect, provided herein is an antibody that specifically binds to human CTLA-4, gemcitabine, and nab-paclitaxel for use in the manufacture of a medicament for the treatment of pancreatic cancer, wherein the treatment is performed according to any one of the methods disclosed herein.

In an aspect, provided herein is a use of an antibody that specifically binds to human CTLA-4, gemcitabine, and nab-paclitaxel for the treatment of pancreatic cancer, wherein the treatment is performed according to any one of the methods disclosed herein.

In an aspect, provided herein is gemcitabine, nab-paclitaxel, and an antibody that specifically binds to human CTLA-4, for use in the treatment of pancreatic cancer, wherein the treatment is performed according to any one of the methods disclosed herein.

In an aspect, provided herein is gemcitabine, nab-paclitaxel, and an antibody that specifically binds to human CTLA-4, for use in the manufacture of a medicament for the treatment of pancreatic cancer, wherein the treatment is performed according to any one of the methods disclosed herein.

In an aspect, provided herein is a use of gemcitabine, nab-paclitaxel, and an antibody that specifically binds to human CTLA-4, for the treatment of pancreatic cancer, wherein the treatment is performed according to any one of the methods disclosed herein.

In an aspect, provided herein is a method of treating a subject with cancer that has metastasized to the liver, the method comprising administering to the subject gemcitabine, nab-paclitaxel, and an antibody that specifically binds to CTLA-4.

In an embodiment, the subject has pancreatic cancer.

In an embodiment, the antibody comprises one or more mutations in the Fc region to increase binding to FcγRIIA and/or FcγRIIIA. In an embodiment, the antibody comprises a human IgG1 Fc region comprising S239D/A330L/1332E mutations, numbered according to the EU numbering system.

In an embodiment, the antibody that specifically binds to human Cytotoxic T-Lymphocyte Antigen 4 (CTLA-4) is administered at a dose of 25 mg to 250 mg, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an embodiment, the antibody is administered at a dose of 25 mg to 200 mg. In an embodiment, the antibody is administered at a dose of about 100-200 mg. In an embodiment, the antibody is administered at a dose of about 150-200 mg. In an embodiment, the antibody is administered at a dose of about 150 mg.

In an embodiment, gemcitabine is administered at a dose of 600 mg/m², 800 mg/m², or 1000 mg/m².

In an embodiment, nab-paclitaxel is administered at a dose of 75 mg/m², 100 mg/m², or 125 mg/m².

In an embodiment, the antibody comprises a VH comprising the amino acid sequence set forth in SEQ ID NO: 7 and a VL comprising the amino acid sequence set forth in SEQ ID NO: 8.

In an embodiment, the antibody comprises botensilimab.

### BRIEF DESCRIPTION OF THE DRAWINGS

FIG. 1A is a graph showing the tumor growth curves of mice treated with either isotype/vehicle control, botensilimab^{ms}, or a conventional anti-CTLA-4 antibody. FIG. 1B is a graph showing the tumor growth curves of mice treated with either isotype/vehicle control, botensilimab^{ms} alone (AGEN1181), nab-paclitaxel (Nab-p)+gemcitabine (Gem), or the combination of botensilimab^{ms} (AGEN1181) and nab-paclitaxel (Nab-p)+gemcitabine (Gem).

FIG. 1C is a graph showing the mouse body weights during the treatment.

FIG. 2A-D is a series of graphs showing the tumor growth curves of individual mice in each treatment group. FIG. 2A is the isotype control treated group, FIG. 2B is the botensilimab^{ms} 15 monotherapy group, FIG. 2C is the nab-paclitaxel+gemcitabine group, and FIG. 2D is the botensilimab^{ms} and nab-paclitaxel+gemcitabine group.

FIG. 3A-D is a series of graphs showing the tumor growth curves of individual mice in each treatment group. FIG. 3A is the isotype control treated group, FIG. 3B is the botensilimab^{ms} monotherapy group, FIG. 3C is the cisplatin+gemcitabine group, and FIG. 3D is the botensilimab^{ms} and cisplatin+gemcitabine group.

FIG. 4A-D is a series of graphs showing the tumor growth curves of individual mice in each treatment group. FIG. 4A is the isotype control treated group, FIG. 4B is the nab-paclitaxel+gemcitabine+cisplatin group, FIG. 4C is the botensilimab^{ms} monotherapy group, and FIG. 4D is the anti-PD-1 antibody monotherapy group.

FIG. 5 is a graph showing the percent change of tumor marker (CA 19-9 or CEA) from baseline over time, in individual patients (n=5) with metastatic pancreatic cancer, who have progressed on FOLFIRINOX, that received 150 mg botensilimab Q6W+gemcitabine (1000 mg/m²)/nab-paclitaxel (125 mg/m²). * indicates the percent change of tumor marker in the fifth patient which showed clinical progression and has been removed from the study.

### DETAILED DESCRIPTION

The instant disclosure is directed to methods for treating pancreatic cancer with an antibody that specifically binds to human Cytotoxic T-Lymphocyte Antigen 4 (CTLA-4), alone or in combination with nab-paclitaxel and/or gemcitabine. Also provided herein are particular methods for administering an antibody that specifically binds to human CTLA-4 that results in a reduction of tumor burden in a subject. In an embodiment, the method comprises a therapeutically effective amount that safely and effectively treats metastatic pancreatic cancer.

### Definitions

As used herein, the terms “antibody” and “antibodies” include full-length antibodies, antigen-binding fragments of full-length antibodies, and molecules comprising antibody CDRs, VH regions, and/or VL regions. Examples of antibodies include, without limitation, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single chain antibodies or single-chain Fvs (scFv), camelized antibodies, affibodies, Fab fragments, F(ab′)2 fragments, disulfide-linked Fvs (sdFv), anti-idiotypic (anti-Id) antibodies (including, e.g., anti-anti-Id antibodies), and antigen-binding fragments of any of the above. In certain embodiments, antibodies described herein refer to polyclonal antibody populations. Antibodies can be of any type (e.g., IgG, IgE, IgM, IgD, IgA, or IgY), any class (e.g., IgG1, IgG2, IgG3, IgG4, IgAQ1, or IgA2), or any subclass (e.g., IgG2a or IgG2b) of immunoglobulin molecule. In certain embodiments, antibodies described herein are IgG antibodies, or a class (e.g., human IgG1 or IgG4) or subclass thereof. In an embodiment, the antibody is a humanized monoclonal antibody. In an embodiment, the antibody is a human monoclonal antibody.

As used herein, the term “CDR” or “complementarity determining region” means the noncontiguous antigen combining sites found within the variable regions of heavy and light chain polypeptides. These particular regions have been described by, for example, Kabat et al., J. Biol. Chem. 252, 6609-6616 (1977) and Kabat et al., Sequences of Proteins of Immunological Interest. (1991), by Chothia et al., J. Mol. Biol. 196:901-917 (1987), and by MacCallum et al., J. Mol. Biol. 262:732-745 (1996), all of which are herein incorporated by reference in their entireties, where the definitions include overlapping or subsets of amino acid residues when compared against each other (see Table 1 below). In certain embodiments, the term “CDR” is a CDR as defined by MacCallum et al., J. Mol. Biol. 262:732-745 (1996) and Martin A. “Protein Sequence and Structure Analysis of Antibody Variable Domains,” in Antibody Engineering, Kontermann and Dübel, eds., Chapter 31, pp. 422-439, Springer-Verlag, Berlin (2001). In certain embodiments, the term “CDR” is a CDR as defined by Kabat et al., J. Biol. Chem. 252, 6609-6616 (1977) and Kabat et al., Sequences of Proteins of Immunological Interest. (1991). In certain embodiments, heavy chain CDRs and light chain CDRs of an antibody are defined using different conventions. In certain embodiments, heavy chain CDRs and/or light chain CDRs are defined by performing structural analysis of an antibody and identifying residues in the variable region(s) predicted to make contact with an epitope region of a target molecule (e.g., human CTLA-4). CDRH1, CDRH2, and CDRH3 denote the heavy chain CDRs, and CDRL1, CDRL2, and CDRL3 denote the light chain CDRs.

TABLE 1
CDR definitions

| | CDR Definitions | | |
|---|---|---|---|
| | Kabat | Chothia | MacCallum |
| VH CDR1 | 31-35 | 26-32 | 30-35 |
| VH CDR2 | 50-65 | 53-55 | 47-58 |
| VH CDR3 | 95-102 | 96-101 | 93-101 |
| VL CDR1 | 24-34 | 26-32 | 30-36 |
| VL CDR2 | 50-56 | 50-52 | 46-55 |
| VL CDR3 | 89-97 | 91-96 | 89-96 |

As used herein, the terms “variable region” and “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable region are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In an embodiment, the variable region is a primate (e.g., non-human primate) variable region. In an embodiment, the variable region comprises rodent or murine CDRs and primate (e.g., non-human primate) framework regions (FRs).

As used herein, the terms “VH” and “VL” refer to antibody heavy and light chain variable regions, respectively, as described in Kabat et al., (1991) Sequences of Proteins of Immunological Interest (NIH Publication No. 91-3242, Bethesda), which is herein incorporated by reference in its entirety.

As used herein, the term “constant region” is common in the art. The constant region is an antibody portion, e.g., a carboxyl terminal portion of a light and/or heavy chain, which is not directly involved in binding of an antibody to antigen, but which can exhibit various effector functions, such as interaction with an Fc receptor (e.g., Fc gamma receptor).

As used herein, the term “heavy chain” when used in reference to an antibody can refer to any distinct type, e.g., alpha (α), delta (δ), epsilon (ε), gamma (γ), and mu (μ), based on the amino acid sequence of the constant region, which give rise to IgA, IgD, IgE, IgG, and IgM classes of antibodies, respectively, including subclasses of IgG, e.g., IgG1, IgG2, IgG3, and IgG4.

As used herein, the term “light chain” when used in reference to an antibody can refer to any distinct type, e.g., kappa (κ) or lambda (λ), based on the amino acid sequence of the constant region. Light chain amino acid sequences are well known in the art. In an embodiment, the light chain is a human light chain.

As used herein, the terms “specifically binds,” “specifically recognizes,” “immunospecifically binds,” and “immunospecifically recognizes” are analogous terms in the context of antibodies and refer to molecules that bind to an antigen (e.g., epitope or immune complex) as such binding is understood by one skilled in the art. For example, a molecule that specifically binds to an antigen can bind to other peptides or polypeptides, generally with lower affinity as determined by, e.g., immunoassays, BIAcore®, KinExA 3000 instrument (Sapidyne Instruments, Boise, ID), or other assays known in the art. In an embodiment, molecules that specifically bind to an antigen bind to the antigen with a KA that is at least 2 logs (e.g., factors of 10), 2.5 logs, 3 logs, 4 logs or greater than the KA when the molecules bind non-specifically to another antigen.

As used herein, the term “EU numbering system” refers to the EU numbering convention for the constant regions of an antibody, as described in Edelman G. M. et al., Proc. Natl. Acad. USA, 63, 78-85 (1969) and Kabat et al., Sequences of Proteins of Immunological Interest, U.S. Dept. Health and Human Services, 5th edition, 1991, each of which is herein incorporated by reference in its entirety.

As used herein, the term “subject” includes any human or non-human animal. In an embodiment, the subject is a human.

As used herein, the term “effective amount” in the context of the administration of a therapy to a subject refers to the amount of a therapy that achieves a desired prophylactic or therapeutic effect.

As used herein, the term “treat,” “treating,” and “treatment” refer to therapeutic or preventative measures described herein. The methods of “treatment” employ administration of an antibody to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “standard of care” refers to the most common treatments prescribed for a particular type of cancer. In an embodiment, the standard of care for metastatic pancreatic cancer includes 5FU, leucovorin, irinotecan, and oxaliplatin (i.e., FOLFIRINOX).

As used herein, the term “targeted therapy” refers to a therapy that inhibits a specific protein. In an embodiment, the targeted therapy inhibits a protein that is known to be important for growth and/or survival of pancreatic cancer cells (e.g., KRAS).

As used herein, the term “cytotoxic therapy” refers to a therapy that blocks or slows cell division. In an embodiment, the cytotoxic therapy kills cancer cells. In an embodiment, the cytotoxic therapy is fluorouracil, capecitabine, oxaliplatin, irinotecan, or trifluridine-tipiracil.

As used herein, the term “tumor burden” refers to the number of cancer cells, the size of a tumor, or the amount of cancer in the body of the subject.

As used herein, the term “about” when referring to a measurable value, such as a dosage, encompasses variations of ±20%, ±15%, ±10%, ±5%, ±1%, or ±0.1% of a given value or range, as are appropriate to perform the methods disclosed herein.

### Anti-CTLA-4 Antibodies

Antibodies that specifically bind to human CTLA-4 (i.e., anti-CTLA-4 antibodies) that are useful in the methods and uses described herein include but are not limited to those listed below.

In an embodiment, the antibody comprises: a heavy chain variable region (VH) comprising the CDRH1, CDRH2, and CDRH3 amino acid sequences of the VH amino acid sequence set forth in SEQ ID NO: 7. In an embodiment, the antibody comprises a heavy chain variable region (VH) comprising the CDRH1, CDRH2, and CDRH3 amino acid sequences of the VH amino acid sequence set forth in SEQ ID NO: 7 and a light chain variable region (VL) comprising the CDRL1, CDRL2, and CDRL3 amino acid sequences of the VL amino acid sequence set forth in SEQ ID NO: 8.

In an embodiment, the antibody comprises the CDRH1, CDRH2, and CDRH3 amino acid sequences set forth in SEQ ID NO: 1, 2, and 3, respectively. In an embodiment, the antibody comprises the CDRH1, CDRH2, CDRH3, CDRL1, CDRL2, and CDRL3 amino acid sequences set forth in SEQ ID NOs: 1, 2, 3, 4, 5, and 6, respectively.

In an embodiment, the antibody comprises a VH comprising the amino acid sequence set forth in SEQ ID NO: 7. In an embodiment, the antibody comprises: a VH comprising the amino acid sequence set forth in SEQ ID NO: 7; and a VL comprising an amino acid sequence which is at least 90%, 95%, 96%, 97%, 98%, 99%, or 100% identical to the amino acid sequence set forth in SEQ ID NO: 8.

In an embodiment, the antibody comprises a VH comprising the amino acid sequence set forth in SEQ ID NO: 7 and a VL comprising the amino acid sequence set forth in SEQ ID NO: 8.

In an embodiment, the antibody comprises a heavy chain constant region selected from the group consisting of human IgG1, IgG2, IgG3, IgG4, IgA1, and IgA2. In an embodiment, the heavy chain constant region is IgG1. In an embodiment, the heavy chain constant region is IgG2. In an embodiment, the antibody comprises a light chain constant region selected from the group consisting of a human kappa light chain constant region and a human lambda light chain constant region.

In an embodiment, the antibody comprises an IgG₁ heavy chain constant region. In an embodiment, the amino acid sequence of the IgG₁ heavy chain constant region comprises S239D/1332E mutations, numbered according to the EU numbering system. In an embodiment, the amino acid sequence of the IgG₁ heavy chain constant region comprises S239D/A330L/1332E mutations, numbered according to the EU numbering system. In an embodiment, the amino acid sequence of the IgG₁ heavy chain constant region comprises L235V/F243L/R292P/Y300L/P396L mutations, numbered according to the EU numbering system. In an embodiment, the IgG₁ heavy chain constant region is afucosylated IgG₁

In an embodiment, the antibody comprises a heavy chain comprising the amino acid sequence set forth in SEQ ID NO: 9. In an embodiment, the antibody comprises a heavy chain comprising the amino acid sequence set forth in SEQ ID NO: 9 and a light chain comprising an amino acid sequence which is at least 90%, 95%, 96%, 97%, 98%, 99%, or 100% identical to the amino acid sequence set forth in SEQ ID NO: 10.

In an embodiment, the antibody comprises a heavy chain comprising the amino acid sequence set forth in SEQ ID NO: 9 and a light chain comprising the amino acid sequence set forth in SEQ ID NO: 10. In an embodiment, the amino acid sequence of the heavy chain consists of the amino acid sequence set forth in SEQ ID NO: 9 and the amino acid sequence of the light chain consists of the amino acid sequence set forth in SEQ ID NO: 10.

In an embodiment the antibody is botensilimab (a.k.a. AGEN1181), the amino acid sequences of which are provided in Table 2 below.

TABLE 2
Amino acid sequences of botensilimab

| | SEQ | |
|---|---|---|
| Descrip- | ID | |
| tion | NO: | Amino acid sequence |
| CDRH1 | 1 | SYSMN |
| CDRH2 | 2 | SISSSSSYIYYAESVKG |
| CDRH3 | 3 | VGLFGPFDI |
| CDRL1 | 4 | RASQSVSRYLG |
| CDRL2 | 5 | GASTRAT |
| CDRL3 | 6 | QQYGSSPWT |
| VH | 7 | EVQLVESGGGLVKPGGSLRLSCAASGFT |
| | | FSSYSMNWVRQAPGKGLEWVSSISSSSS |
| | | YIYYAESVKGRFTISRDNAKNSLYLQMN |
| | | SLRAEDTAVYYCARVGLFGPFDIWGQGT |
| | | LVTVSS |
| VL | 8 | EIVLTQSPGTLSLSPGERATLSCRASQS |
| | | VSRYLGWYQQKPGQAPRLLIYGASTRAT |
| | | GIPDRFSGSGSGTDFTLTITRLEPEDFA |
| | | VYYCQQYGSSPWTFGQGTKVEIK |
| Heavy | 9 | EVQLVESGGGLVKPGGSLRLSCAASGFT |
| chain | | FSSYSMNWVRQAPGKGLEWVSSISSSSS |
| | | YIYYAESVKGRFTISRDNAKNSLYLQMN |
| | | SLRAEDTAVYYCARVGLFGPFDIWGQGT |
| | | LVTVSSASTKGPSVFPLAPSSKSTSGGT |
| | | AALGCLVKDYFPEPVTVSWNSGALTSGV |
| | | HTFPAVLQSSGLYSLSSVVTVPSSSLGT |
| | | QTYICNVNHKPSNTKVDKRVEPKSCDKT |
| | | HTCPPCPAPELLGGPDVFLFPPKPKDTL |
| | | MISRTPEVTCVVVDVSHEDPEVKFNWYV |
| | | DGVEVHNAKTKPREEQYNSTYRVVSVLT |
| | | VLHQDWLNGKEYKCKVSNKALPLPEEKT |
| | | ISKAKGQPREPQVYTLPPSREEMTKNQV |
| | | SLTCLVKGFYPSDIAVEWESNGQPENNY |
| | | KTTPPVLDSDGSFFLYSKLTVDKSRWQQ |
| | | GNVFSCSVMHEALHNHYTQKSLSLSPG |
| Light | 10 | EIVLTQSPGTLSLSPGERATLSCRASQS |
| chain | | VSRYLGWYQQKPGQAPRLLIYGASTRAT |
| | | GIPDRFSGSGSGTDFTLTITRLEPEDFA |
| | | VYYCQQYGSSPWTFGQGTKVEIKRTVAA |
| | | PSVFIFPPSDEQLKSGTASVVCLLNNFY |
| | | PREAKVQWKVDNALQSGNSQESVTEQDS |
| | | KDSTYSLSSTLTLSKADYEKHKVYACEV |
| | | THQGLSSPVTKSFNRGEC |

### Methods of Treatment

The instant disclosure demonstrates that antibodies that specifically bind to human CTLA-4 (e.g., botensilimab) are highly effective in treating pancreatic cancer. The instant disclosure also demonstrates that antibodies that specifically bind to human CTLA-4 (e.g., botensilimab) are highly effective in treating metastatic pancreatic adenocarcinoma that has progressed on prior 5FU+Leucovorin+Irinotecan+Oxaliplatin (FOLFIRINOX) therapy. Accordingly, the instant disclosure is broadly directed to methods for treating pancreatic cancer with an antibody that specifically binds to human CTLA-4 as a monotherapy or in combination with nab-paclitaxel and gemcitabine.

In an aspect, provided herein is a method of treating pancreatic cancer in a subject in need thereof, the method comprising administering to the subject an effective amount of an antibody that specifically binds to human CTLA-4, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an aspect, provided herein is a method of enhancing the activation of T cells in a subject who has pancreatic cancer, the method comprising administering to the subject an effective amount of an antibody that specifically binds to human CTLA-4, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an aspect, provided herein is a method of treating pancreatic cancer in a subject in need thereof, the method comprising administering to the subject an antibody that specifically binds to human Cytotoxic T-Lymphocyte Antigen 4 (CTLA-4) at a dose of about 5 mg to about 250 mg, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an aspect, provided herein is a method of enhancing the activation of T cells in a subject who has pancreatic cancer, the method comprising administering to the subject an antibody that specifically binds to human CTLA-4 at a dose of about 5 mg to about 250 mg, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an aspect, provided herein is a method of treating pancreatic cancer in a subject in need thereof, the method comprising administering to the subject an antibody that specifically binds to human Cytotoxic T-Lymphocyte Antigen 4 (CTLA-4) at a dose of 5 mg to 250 mg, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an aspect, provided herein is a method of enhancing the activation of T cells in a subject who has pancreatic cancer, the method comprising administering to the subject an antibody that specifically binds to human CTLA-4 at a dose of 5 mg to 250 mg, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an embodiment, provided herein is a method of treating pancreatic cancer in a subject in need thereof, the method comprising administering to the subject an antibody that specifically binds to human Cytotoxic T-Lymphocyte Antigen 4 (CTLA-4) at a dose of about 5 mg to about 200 mg, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an embodiment, provided herein is a method of enhancing the activation of T cells in a subject who has pancreatic cancer, the method comprising administering to the subject an antibody that specifically binds to human CTLA-4 at a dose of about 5 mg to about 200 mg, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an embodiment, provided herein is a method of treating pancreatic cancer in a subject in need thereof, the method comprising administering to the subject an antibody that specifically binds to human Cytotoxic T-Lymphocyte Antigen 4 (CTLA-4) at a dose of 5 mg to 200 mg, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an embodiment, provided herein is a method of enhancing the activation of T cells in a subject who has pancreatic cancer, the method comprising administering to the subject an antibody that specifically binds to human CTLA-4 at a dose of 5 mg to 200 mg, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an embodiment, the antibody is administered at a dose of about 25 mg to about 250 mg. In an embodiment, the antibody is administered at a dose of about 25 mg to about 200 mg. In an embodiment, the antibody is administered at a dose of about 25 mg to about 150 mg. In an embodiment, the antibody is administered at a dose of about 50 mg to about 150 mg. In an embodiment, the antibody is administered at a dose of about 75 mg to about 150 mg.

In an embodiment, the antibody is administered at a dose of 25 mg to 250 mg. In an embodiment, the antibody is administered at a dose of 25 mg to 200 mg. In an embodiment, the antibody is administered at a dose of 50 mg to 175 mg. In an embodiment, the antibody is administered at a dose of 25 mg to 150 mg. In an embodiment, the antibody is administered at a dose of 50 mg to 150 mg. In an embodiment, the antibody is administered at a dose of 75 mg to 150 mg.

In an embodiment, the antibody is administered at a dose of about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 175 mg, about 200 mg, about 225 mg, or about 250 mg.

In an embodiment, the antibody is administered at a dose of 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 175 mg, 200 mg, 225 mg, or 250 mg.

In an embodiment, the antibody is administered intravenously. In an embodiment, the antibody is administered intratumorally.

In an embodiment, the antibody is administered by intravenous infusion over about 30 minutes. In an embodiment, the antibody is administered by intravenous infusion over about 45 minutes. In an embodiment, the antibody is administered by intravenous infusion over about 60 minutes. In an embodiment, the antibody is administered by intravenous infusion over about 90 minutes.

In an embodiment, the antibody is administered about once weekly. In an embodiment, the antibody is administered about once every 2 weeks. In an embodiment, the antibody is administered about once every 3 weeks. In an embodiment, the antibody is administered about once every 4 weeks. In an embodiment, the antibody is administered about once every 5 weeks. In an embodiment, the antibody is administered about once every 6 weeks. In an embodiment, the antibody is administered about once every 7 weeks. In an embodiment, the antibody is administered about once every 8 weeks.

In an embodiment, the antibody is administered once weekly. In an embodiment, the antibody is administered once every 2 weeks. In an embodiment, the antibody is administered once every 3 weeks. In an embodiment, the antibody is administered once every 4 weeks. In an embodiment, the antibody is administered once every 5 weeks. In an embodiment, the antibody is administered once every 6 weeks. In an embodiment, the antibody is administered once every 7 weeks. In an embodiment, the antibody is administered once every 8 weeks.

In an embodiment, the antibody is administered intravenously at a dose of about 25 mg once every 6 weeks. In an embodiment, the antibody is administered intravenously at a dose of about 50 mg once every 6 weeks. In an embodiment, the antibody is administered intravenously at a dose of about 75 mg once every 6 weeks. In an embodiment, the antibody is administered intravenously at a dose of about 100 mg once every 6 weeks. In an embodiment, the antibody is administered intravenously at a dose of about 125 mg once every 6 weeks. In In an embodiment, the antibody is administered intravenously at a dose of about 150 mg once every 6 weeks. In an embodiment, the antibody is administered intravenously at a dose of about 175 mg once every 6 weeks. In an embodiment, the antibody is administered intravenously at a dose of about 200 mg once every 6 weeks. In an embodiment, the antibody is administered intravenously at a dose of about 225 mg once every 6 weeks. In an embodiment, the antibody is administered intravenously at a dose of about 250 mg once every 6 weeks.

In an embodiment, the antibody is administered intravenously at a dose of 25 mg once every 6 weeks. In an embodiment, the antibody is administered intravenously at a dose of 50 mg once every 6 weeks. In an embodiment, the antibody is administered intravenously at a dose of 75 mg once every 6 weeks. In an embodiment, the antibody is administered intravenously at a dose of 100 mg once every 6 weeks. In an embodiment, the antibody is administered intravenously at a dose of 125 mg once every 6 weeks. In an embodiment, the antibody is administered intravenously at a dose of 150 mg once every 6 weeks. In an embodiment, the antibody is administered intravenously at a dose of 175 mg once every 6 weeks. In an embodiment, the antibody is administered intravenously at a dose of 200 mg once every 6 weeks. In an embodiment, the antibody is administered intravenously at a dose of 225 mg once every 6 weeks. In an embodiment, the antibody is administered intravenously at a dose of 250 mg once every 6 weeks.

In an embodiment, the dose is a therapeutically effective amount.

In an embodiment, the pancreatic cancer is pancreatic ductal adenocarcinoma. In an embodiment, the pancreatic cancer is unresectable. In an embodiment, the pancreatic cancer is metastatic. In an embodiment, the pancreatic cancer is relapsed and/or refractory. In an embodiment, the pancreatic cancer has progressed after 5-fluorouracil, leucovorin, irinotecan, and oxaliplatin therapy (i.e., FOLFIRINOX therapy).

In an embodiment, the subject has received at least one prior chemotherapy. In an embodiment, the at least one prior chemotherapy is 5-fluorouracil, leucovorin, irinotecan, or oxaliplatin. In an embodiment, the subject has previously been treated with 5-fluorouracil, leucovorin, irinotecan, and oxaliplatin (i.e., FOLFIRINOX therapy). In an embodiment, the pancreatic cancer relapsed following a standard of care treatment.

In an embodiment, the method further comprises administering nab-paclitaxel to the subject. In an embodiment, nab-paclitaxel is administered at a dose of about 50 mg/m² to about 200 mg/m². In an embodiment, nab-paclitaxel is administered at a dose of about 75 mg/m² to about 125 mg/m². In an embodiment, nab-paclitaxel is administered at a dose of about 75 mg/m², about 100 mg/m², or about 125 mg/m². In an embodiment, nab-paclitaxel is administered once every week. In an embodiment, nab-paclitaxel is administered on days 1, 8, and 15 of a 6 week cycle.

In an embodiment, the method further comprises administering nab-paclitaxel to the subject. In an embodiment, nab-paclitaxel is administered at a dose of 75 mg/m² to 200 mg/m². In an embodiment, nab-paclitaxel is administered at a dose of 75 mg/m² to 125 mg/m². In an embodiment, nab-paclitaxel is administered at a dose of 75 mg/m², 100 mg/m², or 125 mg/m². In an embodiment, nab-paclitaxel is administered once every week. In an embodiment, nab-paclitaxel is administered on days 1, 8, and 15 of a 6 week cycle.

In an embodiment, the method further comprises administering gemcitabine to the subject. In an embodiment, gemcitabine is administered at a dose of about 500 mg/m² to about 1500 mg/m². In an embodiment, gemcitabine is administered at a dose of about 600 mg/m² to about 1000 mg/m². In an embodiment, gemcitabine is administered at a dose of about 600 mg/m², about 800 mg/m², or about 1000 mg/m². In an embodiment, gemcitabine is administered once every week. In an embodiment, gemcitabine is administered on days 1, 8, and 15 of a 6 week cycle.

In an embodiment, the method further comprises administering gemcitabine to the subject. In an embodiment, gemcitabine is administered at a dose of 500 mg/m² to 1500 mg/m². In an embodiment, gemcitabine is administered at a dose of 600 mg/m² to 1000 mg/m². In an embodiment, gemcitabine is administered at a dose of 600 mg/m², 800 mg/m², or 1000 mg/m². In an embodiment, gemcitabine is administered once every week. In an embodiment, gemcitabine is administered on days 1, 8, and 15 of a 6 week cycle.

In an embodiment, the antibody is administered intravenously at a dose of 25 mg once every 6 weeks, nab-paclitaxel is administered at a dose of 75 mg/m², and gemcitabine is administered at a dose of 600 mg/m². In an embodiment, the antibody is administered intravenously at a dose of 50 mg once every 6 weeks, nab-paclitaxel is administered at a dose of 75 mg/m², and gemcitabine is administered at a dose of 600 mg/m². In an embodiment, the antibody is administered intravenously at a dose of 75 mg once every 6 weeks, nab-paclitaxel is administered at a dose of 75 mg/m², and gemcitabine is administered at a dose of 600 mg/m². In an embodiment, the antibody is administered intravenously at a dose of 100 mg once every 6 weeks, nab-paclitaxel is administered at a dose of 75 mg/m², and gemcitabine is administered at a dose of 600 mg/m². In an embodiment, the antibody is administered intravenously at a dose of 125 mg once every 6 weeks, nab-paclitaxel is administered at a dose of 75 mg/m², and gemcitabine is administered at a dose of 600 mg/m². In an embodiment, the antibody is administered intravenously at a dose of 150 mg once every 6 weeks, nab-paclitaxel is administered at a dose of 75 mg/m², and gemcitabine is administered at a dose of 600 mg/m². In an embodiment, the antibody is administered intravenously at a dose of 175 mg once every 6 weeks, nab-paclitaxel is administered at a dose of 75 mg/m², and gemcitabine is administered at a dose of 600 mg/m². In an embodiment, the antibody is administered intravenously at a dose of 200 mg once every 6 weeks, nab-paclitaxel is administered at a dose of 75 mg/m², and gemcitabine is administered at a dose of 600 mg/m². In an embodiment, the antibody is administered intravenously at a dose of 225 mg once every 6 weeks, nab-paclitaxel is administered at a dose of 75 mg/m², and gemcitabine is administered at a dose of 600 mg/m². In an embodiment, the antibody is administered intravenously at a dose of 250 mg once every 6 weeks, nab-paclitaxel is administered at a dose of 75 mg/m², and gemcitabine is administered at a dose of 600 mg/m².

In an embodiment, the antibody is administered intravenously at a dose of 25 mg, nab-paclitaxel is administered at a dose of 100 mg/m², and gemcitabine is administered at a dose of 600 mg/m². In an embodiment, the antibody is administered intravenously at a dose of 50 mg, nab-paclitaxel is administered at a dose of 100 mg/m², and gemcitabine is administered at a dose of 600 mg/m². In an embodiment, the antibody is administered intravenously at a dose of 75 mg, nab-paclitaxel is administered at a dose of 100 mg/m², and gemcitabine is administered at a dose of 600 mg/m². In an embodiment, the antibody is administered intravenously at a dose of 100 mg, nab-paclitaxel is administered at a dose of 100 mg/m², and gemcitabine is administered at a dose of 600 mg/m². In an embodiment, the antibody is administered intravenously at a dose of 125 mg, nab-paclitaxel is administered at a dose of 100 mg/m², and gemcitabine is administered at a dose of 600 mg/m². In an embodiment, the antibody is administered intravenously at a dose of 150 mg, nab-paclitaxel is administered at a dose of 100 mg/m², and gemcitabine is administered at a dose of 600 mg/m². In an embodiment, the antibody is administered intravenously at a dose of 175 mg, nab-paclitaxel is administered at a dose of 100 mg/m², and gemcitabine is administered at a dose of 600 mg/m². In an embodiment, the antibody is administered intravenously at a dose of 200 mg, nab-paclitaxel is administered at a dose of 100 mg/m², and gemcitabine is administered at a dose of 600 mg/m². In an embodiment, the antibody is administered intravenously at a dose of 225 mg, nab-paclitaxel is administered at a dose of 100 mg/m², and gemcitabine is administered at a dose of 600 mg/m². In an embodiment, the antibody is administered intravenously at a dose of 250 mg, nab-paclitaxel is administered at a dose of 100 mg/m², and gemcitabine is administered at a dose of 600 mg/m².

In an embodiment, the antibody is administered intravenously at a dose of 25 mg, nab-paclitaxel is administered at a dose of 125 mg/m², and gemcitabine is administered at a dose of 600 mg/m². In an embodiment, the antibody is administered intravenously at a dose of 50 mg, nab-paclitaxel is administered at a dose of 125 mg/m², and gemcitabine is administered at a dose of 600 mg/m². In an embodiment, the antibody is administered intravenously at a dose of 75 mg, nab-paclitaxel is administered at a dose of 125 mg/m², and gemcitabine is administered at a dose of 600 mg/m². In an embodiment, the antibody is administered intravenously at a dose of 100 mg, nab-paclitaxel is administered at a dose of 125 mg/m², and gemcitabine is administered at a dose of 600 mg/m². In an embodiment, the antibody is administered intravenously at a dose of 125 mg, nab-paclitaxel is administered at a dose of 125 mg/m², and gemcitabine is administered at a dose of 600 mg/m². In an embodiment, the antibody is administered intravenously at a dose of 150 mg, nab-paclitaxel is administered at a dose of 125 mg/m², and gemcitabine is administered at a dose of 600 mg/m². In an embodiment, the antibody is administered intravenously at a dose of 175 mg, nab-paclitaxel is administered at a dose of 125 mg/m², and gemcitabine is administered at a dose of 600 mg/m². In an embodiment, the antibody is administered intravenously at a dose of 200 mg, nab-paclitaxel is administered at a dose of 125 mg/m², and gemcitabine is administered at a dose of 600 mg/m². In an embodiment, the antibody is administered intravenously at a dose of 225 mg, nab-paclitaxel is administered at a dose of 125 mg/m², and gemcitabine is administered at a dose of 600 mg/m². In an embodiment, the antibody is administered intravenously at a dose of 250 mg, nab-paclitaxel is administered at a dose of 125 mg/m², and gemcitabine is administered at a dose of 600 mg/m².

In an embodiment, the antibody is administered intravenously at a dose of 25 mg once every 6 weeks, nab-paclitaxel is administered at a dose of 75 mg/m², and gemcitabine is administered at a dose of 800 mg/m². In an embodiment, the antibody is administered intravenously at a dose of 50 mg once every 6 weeks, nab-paclitaxel is administered at a dose of 75 mg/m², and gemcitabine is administered at a dose of 800 mg/m². In an embodiment, the antibody is administered intravenously at a dose of 75 mg once every 6 weeks, nab-paclitaxel is administered at a dose of 75 mg/m², and gemcitabine is administered at a dose of 800 mg/m². In an embodiment, the antibody is administered intravenously at a dose of 100 mg once every 6 weeks, nab-paclitaxel is administered at a dose of 75 mg/m², and gemcitabine is administered at a dose of 800 mg/m². In an embodiment, the antibody is administered intravenously at a dose of 125 mg once every 6 weeks, nab-paclitaxel is administered at a dose of 75 mg/m², and gemcitabine is administered at a dose of 800 mg/m². In an embodiment, the antibody is administered intravenously at a dose of 150 mg once every 6 weeks, nab-paclitaxel is administered at a dose of 75 mg/m², and gemcitabine is administered at a dose of 800 mg/m². In an embodiment, the antibody is administered intravenously at a dose of 175 mg once every 6 weeks, nab-paclitaxel is administered at a dose of 75 mg/m², and gemcitabine is administered at a dose of 800 mg/m². In an embodiment, the antibody is administered intravenously at a dose of 200 mg once every 6 weeks, nab-paclitaxel is administered at a dose of 75 mg/m², and gemcitabine is administered at a dose of 800 mg/m². In an embodiment, the antibody is administered intravenously at a dose of 225 mg once every 6 weeks, nab-paclitaxel is administered at a dose of 75 mg/m², and gemcitabine is administered at a dose of 800 mg/m². In an embodiment, the antibody is administered intravenously at a dose of 250 mg once every 6 weeks, nab-paclitaxel is administered at a dose of 75 mg/m², and gemcitabine is administered at a dose of 800 mg/m².

In an embodiment, the antibody is administered intravenously at a dose of 25 mg, nab-paclitaxel is administered at a dose of 100 mg/m², and gemcitabine is administered at a dose of 800 mg/m². In an embodiment, the antibody is administered intravenously at a dose of 50 mg, nab-paclitaxel is administered at a dose of 100 mg/m², and gemcitabine is administered at a dose of 800 mg/m². In an embodiment, the antibody is administered intravenously at a dose of 75 mg, nab-paclitaxel is administered at a dose of 100 mg/m², and gemcitabine is administered at a dose of 800 mg/m². In an embodiment, the antibody is administered intravenously at a dose of 100 mg, nab-paclitaxel is administered at a dose of 100 mg/m², and gemcitabine is administered at a dose of 800 mg/m². In an embodiment, the antibody is administered intravenously at a dose of 125 mg, nab-paclitaxel is administered at a dose of 100 mg/m², and gemcitabine is administered at a dose of 800 mg/m². In an embodiment, the antibody is administered intravenously at a dose of 150 mg, nab-paclitaxel is administered at a dose of 100 mg/m², and gemcitabine is administered at a dose of 800 mg/m². In an embodiment, the antibody is administered intravenously at a dose of 175 mg, nab-paclitaxel is administered at a dose of 100 mg/m², and gemcitabine is administered at a dose of 800 mg/m². In an embodiment, the antibody is administered intravenously at a dose of 200 mg, nab-paclitaxel is administered at a dose of 100 mg/m², and gemcitabine is administered at a dose of 800 mg/m². In an embodiment, the antibody is administered intravenously at a dose of 225 mg, nab-paclitaxel is administered at a dose of 100 mg/m², and gemcitabine is administered at a dose of 800 mg/m². In an embodiment, the antibody is administered intravenously at a dose of 250 mg, nab-paclitaxel is administered at a dose of 100 mg/m², and gemcitabine is administered at a dose of 800 mg/m².

In an embodiment, the antibody is administered intravenously at a dose of 25 mg, nab-paclitaxel is administered at a dose of 125 mg/m², and gemcitabine is administered at a dose of 800 mg/m². In an embodiment, the antibody is administered intravenously at a dose of 50 mg, nab-paclitaxel is administered at a dose of 125 mg/m², and gemcitabine is administered at a dose of 800 mg/m². In an embodiment, the antibody is administered intravenously at a dose of 75 mg, nab-paclitaxel is administered at a dose of 125 mg/m², and gemcitabine is administered at a dose of 800 mg/m². In an embodiment, the antibody is administered intravenously at a dose of 125 mg, nab-paclitaxel is administered at a dose of 125 mg/m², and gemcitabine is administered at a dose of 800 mg/m². In an embodiment, the antibody is administered intravenously at a dose of 100 mg, nab-paclitaxel is administered at a dose of 125 mg/m², and gemcitabine is administered at a dose of 800 mg/m². In an embodiment, the antibody is administered intravenously at a dose of 150 mg, nab-paclitaxel is administered at a dose of 125 mg/m², and gemcitabine is administered at a dose of 800 mg/m². In an embodiment, the antibody is administered intravenously at a dose of 175 mg, nab-paclitaxel is administered at a dose of 125 mg/m², and gemcitabine is administered at a dose of 800 mg/m². In an embodiment, the antibody is administered intravenously at a dose of 200 mg, nab-paclitaxel is administered at a dose of 125 mg/m², and gemcitabine is administered at a dose of 800 mg/m². In an embodiment, the antibody is administered intravenously at a dose of 225 mg, nab-paclitaxel is administered at a dose of 125 mg/m², and gemcitabine is administered at a dose of 800 mg/m². In an embodiment, the antibody is administered intravenously at a dose of 250 mg, nab-paclitaxel is administered at a dose of 125 mg/m², and gemcitabine is administered at a dose of 800 mg/m².

In an embodiment, the antibody is administered intravenously at a dose of 25 mg once every 6 weeks, nab-paclitaxel is administered at a dose of 75 mg/m², and gemcitabine is administered at a dose of 1000 mg/m². In an embodiment, the antibody is administered intravenously at a dose of 50 mg once every 6 weeks, nab-paclitaxel is administered at a dose of 75 mg/m², and gemcitabine is administered at a dose of 1000 mg/m². In an embodiment, the antibody is administered intravenously at a dose of 75 mg once every 6 weeks, nab-paclitaxel is administered at a dose of 75 mg/m², and gemcitabine is administered at a dose of 1000 mg/m². In an embodiment, the antibody is administered intravenously at a dose of 100 mg once every 6 weeks, nab-paclitaxel is administered at a dose of 75 mg/m², and gemcitabine is administered at a dose of 1000 mg/m². In an embodiment, the antibody is administered intravenously at a dose of 125 mg once every 6 weeks, nab-paclitaxel is administered at a dose of 75 mg/m², and gemcitabine is administered at a dose of 1000 mg/m². In an embodiment, the antibody is administered intravenously at a dose of 150 mg once every 6 weeks, nab-paclitaxel is administered at a dose of 75 mg/m², and gemcitabine is administered at a dose of 1000 mg/m². In an embodiment, the antibody is administered intravenously at a dose of 175 mg once every 6 weeks, nab-paclitaxel is administered at a dose of 75 mg/m², and gemcitabine is administered at a dose of 1000 mg/m². In an embodiment, the antibody is administered intravenously at a dose of 200 mg once every 6 weeks, nab-paclitaxel is administered at a dose of 75 mg/m², and gemcitabine is administered at a dose of 1000 mg/m². In an embodiment, the antibody is administered intravenously at a dose of 225 mg once every 6 weeks, nab-paclitaxel is administered at a dose of 75 mg/m², and gemcitabine is administered at a dose of 1000 mg/m². In an embodiment, the antibody is administered intravenously at a dose of 250 mg once every 6 weeks, nab-paclitaxel is administered at a dose of 75 mg/m², and gemcitabine is administered at a dose of 1000 mg/m².

In an embodiment, the antibody is administered intravenously at a dose of 25 mg, nab-paclitaxel is administered at a dose of 100 mg/m², and gemcitabine is administered at a dose of 1000 mg/m². In an embodiment, the antibody is administered intravenously at a dose of 50 mg, nab-paclitaxel is administered at a dose of 100 mg/m², and gemcitabine is administered at a dose of 1000 mg/m². In an embodiment, the antibody is administered intravenously at a dose of 75 mg, nab-paclitaxel is administered at a dose of 100 mg/m², and gemcitabine is administered at a dose of 1000 mg/m². In an embodiment, the antibody is administered intravenously at a dose of 100 mg, nab-paclitaxel is administered at a dose of 100 mg/m², and gemcitabine is administered at a dose of 1000 mg/m². In an embodiment, the antibody is administered intravenously at a dose of 125 mg, nab-paclitaxel is administered at a dose of 100 mg/m², and gemcitabine is administered at a dose of 1000 mg/m². In an embodiment, the antibody is administered intravenously at a dose of 150 mg, nab-paclitaxel is administered at a dose of 100 mg/m², and gemcitabine is administered at a dose of 1000 mg/m². In an embodiment, the antibody is administered intravenously at a dose of 175 mg, nab-paclitaxel is administered at a dose of 100 mg/m², and gemcitabine is administered at a dose of 1000 mg/m². In an embodiment, the antibody is administered intravenously at a dose of 200 mg, nab-paclitaxel is administered at a dose of 100 mg/m², and gemcitabine is administered at a dose of 1000 mg/m². In an embodiment, the antibody is administered intravenously at a dose of 225 mg, nab-paclitaxel is administered at a dose of 100 mg/m², and gemcitabine is administered at a dose of 1000 mg/m². In an embodiment, the antibody is administered intravenously at a dose of 250 mg, nab-paclitaxel is administered at a dose of 100 mg/m², and gemcitabine is administered at a dose of 1000 mg/m².

In an embodiment, the antibody is administered intravenously at a dose of 25 mg, nab-paclitaxel is administered at a dose of 125 mg/m², and gemcitabine is administered at a dose of 1000 mg/m². In an embodiment, the antibody is administered intravenously at a dose of 50 mg, nab-paclitaxel is administered at a dose of 125 mg/m², and gemcitabine is administered at a dose of 1000 mg/m². In an embodiment, the antibody is administered intravenously at a dose of 75 mg, nab-paclitaxel is administered at a dose of 125 mg/m², and gemcitabine is administered at a dose of 1000 mg/m². In an embodiment, the antibody is administered intravenously at a dose of 100 mg, nab-paclitaxel is administered at a dose of 125 mg/m², and gemcitabine is administered at a dose of 1000 mg/m². In an embodiment, the antibody is administered intravenously at a dose of 125 mg, nab-paclitaxel is administered at a dose of 125 mg/m², and gemcitabine is administered at a dose of 1000 mg/m². In an embodiment, the antibody is administered intravenously at a dose of 150 mg, nab-paclitaxel is administered at a dose of 125 mg/m², and gemcitabine is administered at a dose of 1000 mg/m². In an embodiment, the antibody is administered intravenously at a dose of 175 mg, nab-paclitaxel is administered at a dose of 125 mg/m², and gemcitabine is administered at a dose of 1000 mg/m². In an embodiment, the antibody is administered intravenously at a dose of 200 mg, nab-paclitaxel is administered at a dose of 125 mg/m², and gemcitabine is administered at a dose of 1000 mg/m². In an embodiment, the antibody is administered intravenously at a dose of 225 mg, nab-paclitaxel is administered at a dose of 125 mg/m², and gemcitabine is administered at a dose of 1000 mg/m². In an embodiment, the antibody is administered intravenously at a dose of 250 mg, nab-paclitaxel is administered at a dose of 125 mg/m², and gemcitabine is administered at a dose of 1000 mg/m².

In an embodiment of any one of the methods disclosed herein, the antibody is administered on day 1 of a 6 week cycle and nab-paclitaxel and gemcitabine are administered on days 1, 8, and 15 of the 6 week cycle. In an embodiment, the antibody is administered to the subject before nab-paclitaxel and gemcitabine. In an embodiment, nab-paclitaxel is administered before gemcitabine. In an embodiment, nab-paclitaxel is administered intravenously. In an embodiment, gemcitabine is administered intravenously.

In an embodiment, the method comprises two 6 week cycles. In an embodiment, the method comprises three 6 week cycles. In an embodiment, the method comprises four 6 week cycles. In an embodiment, the method comprises five 6 week cycles. In an embodiment, the method comprises five 6 week cycles. In an embodiment, the method comprises six 6 week cycles. In an embodiment, the method comprises seven 6 week cycles. In an embodiment, the method comprises eight 6 week cycles.

In an embodiment, the subject is a human subject. In an embodiment, the method does not comprise administering cisplatin.

In an embodiment, the cancer is refractory to a standard of care treatment. In an embodiment, the standard of care treatment is chemotherapy or radiation. In an embodiment, the standard of care treatment is 5-fluorouracil, leucovorin, irinotecan, and/or oxaliplatin.

In an embodiment, the method reduces tumor size in the subject. In an embodiment, the method increases T-cell activation in the subject. In an embodiment, the method reduces the level of CA 19-9, CA 125, or CEA in the subject.

In an embodiment, before administration of the antibody the subject has measurable disease on baseline imaging per RECIST 1.1. In an embodiment, before administration of the antibody the subject has an Eastern Cooperative Oncology Group performance status (PS) 0-1. In an embodiment, before administration of the antibody the subject has a predicted life expectancy of ≥12 weeks.

In an embodiment, before administration of the antibody the subject has adequate organ function as defined by one or more of: a) neutrophils≥1500/μL; b) platelets≥100×10³/μL; c) hemoglobin≥9.0 g/dL; d) creatinine clearance ≥30 mL/min as measured or calculated per local institutional standards; e) Aspartate aminotransferase (AST)/alanine aminotransferase (ALT)<3.0×upper limit of normal (ULN); f) direct bilirubin <1.5×ULN (except patients with Gilbert syndrome who must have a total bilirubin level of <3.0×ULN); and/or g) serum albumin≥3.0 g/dL.

In an embodiment, the subject does not have partial or complete bowel obstruction within the last 3 months, signs/symptoms of bowel obstruction, or known radiologic evidence of impending obstruction.

In an embodiment, the subject has not received an immune checkpoint inhibitor therapy prior to administration of the antibody. In an embodiment, the subject has not received more than one chemotherapy regimen prior to administration of the antibody. In an embodiment, the subject does not have a history of central nervous system (CNS) metastases. In an embodiment, the subject does not have a concurrent malignancy that requires treatment or a history of prior malignancy that was active within 2 years prior to administration of the antibody. In an embodiment, the subject has not received a cytotoxic therapy or targeted therapy, within 3 weeks prior to administration of the antibody. In an embodiment, the subject has not received other monoclonal antibody therapy, antibody-drug conjugate therapy, or radioimmunoconjugate therapy, within 4 weeks prior to administration of the antibody. In an embodiment, the subject has not received small molecule tyrosine kinase inhibitor therapy within 2 weeks prior to administration of the antibody.

In an embodiment, the subject does not have refractory ascites defined as requiring 2 or more therapeutic paracenteses within the last 4 weeks or ≥4 times within the last 90 days or ≥1 time within the last 2 weeks prior to administration of the antibody. In an embodiment, the subject does not have clinically significant cardiovascular disease.

In an embodiment, the objective response rate (ORR), duration of response (DOR), disease control rate (DCR), and progression-free survival (PFS) are assessed for a subject according to the Response Evaluation Criteria in Solid Tumors Version 1.1 (RECIST 1.1).

In an embodiment, the method results in a complete response, as defined by RECIST 1.1. In an embodiment, the method results in a partial response, as defined by RECIST 1.1. In an embodiment, the method results in a stable disease, as defined by RECIST 1.1.

In an embodiment, the method results in about a 1, 5, 10, 20, 30, 40, 50, 60, 70, 80, 90, or 100% reduction in tumor burden in the subject. In an embodiment, the method results in no change in tumor burden in the subject. In an embodiment, the method results in about a 1% reduction in tumor burden in the subject. In an embodiment, the method results in about a 5% reduction in tumor burden in the subject. In an embodiment, the method results in about a 10% reduction in tumor burden in the subject. In an embodiment, the method results in about a 20% reduction in tumor burden in the subject. In an embodiment, the method results in about a 30% reduction in tumor burden in the subject. In an embodiment, the method results in about a 40% reduction in tumor burden in the subject. In an embodiment, the method results in about a 50% reduction in tumor burden in the subject. In an embodiment, the method results in about a 60% reduction in tumor burden in the subject. In an embodiment, the method results in about a 70% reduction in tumor burden in the subject. In an embodiment, the method results in about an 80% reduction in tumor burden in the subject. In an embodiment, the method results in about a 90% reduction in tumor burden in the subject. In an embodiment, the method results in about a 100% reduction in tumor burden in the subject.

In an embodiment, the method results in a reduced tumor burden. In an embodiment, the method results in increased survival. In an embodiment, the method results in an increase in overall survival. In an embodiment, the method results in an increase in progression-free survival.

In an aspect, provided herein is an antibody that specifically binds to human CTLA-4 for use in the treatment of pancreatic cancer, wherein the treatment is performed according to any one of the methods disclosed herein.

In an aspect, provided herein is an antibody that specifically binds to human CTLA-4 for use in the manufacture of a medicament for the treatment of pancreatic cancer, wherein the treatment is performed according to any one of the methods disclosed herein.

In an aspect, provided herein is a use of an antibody that specifically binds to human CTLA-4 for the treatment of pancreatic cancer, wherein the treatment is performed according to any one of the methods disclosed herein.

In an aspect, provided herein is an antibody that specifically binds to human CTLA-4, gemcitabine, and nab-paclitaxel for use in the treatment of pancreatic cancer, wherein the treatment is performed according to any one of the methods disclosed herein.

In an aspect, provided herein is an antibody that specifically binds to human CTLA-4, gemcitabine, and nab-paclitaxel for use in the manufacture of a medicament for the treatment of pancreatic cancer, wherein the treatment is performed according to any one of the methods disclosed herein.

In an aspect, provided herein is a use of an antibody that specifically binds to human CTLA-4, gemcitabine, and nab-paclitaxel for the treatment of pancreatic cancer, wherein the treatment is performed according to any one of the methods disclosed herein.

### EXAMPLES

### Example 1—Botensilimab (AGEN1181) in Combination with Nab-paclitaxel and Gemcitabine in a Mouse Model of Pancreatic Cancer

Botensilimab was tested for its ability to inhibit pancreatic cancer growth in a mouse model of pancreatic cancer.

Female C57BL/6J mice (6-8 weeks old) were implanted with KPC (KrasG12D, P53−/−Pdx1-Cre) tumor chunks (˜100 mm³) isolated from KPC tumor-bearing mice. Mice were anesthetized with isoflurane for the implantations.

The mice were then randomized and treated with the indicated antibodies, chemotherapy, or combinations thereof. The antibodies included: isotype control, mouse surrogate of conventional or first-generation anti-CTLA-4 (Clone 9D9 mouse IgG2b), mouse surrogate of botensilimab (botensilimab^{ms}; Clone 9D9 mouse IgG2b.SDALIE) or anti-PD-1 (Clone RMP1-14). Chemotherapy agents included gemcitabine, nab-paclitaxel (Abraxane) and cisplatin.

Antibodies (100 μg/dose) were administered intraperitoneally (i.p), twice a week for three weeks. Mice treated with doublet chemotherapy received gemcitabine (70 mg/kg) intraperitoneally and Abraxane (25 mg/kg) intravenously or gemcitabine (70 mg/kg) intraperitoneally and cisplatin (4 mg/kg) intraperitoneally on days 1 and 4. Mice treated with triplet chemotherapy received gemcitabine (70 mg/kg) intraperitoneally, abraxane (25 mg/kg) intravenously and cisplatin (4 mg/kg) intraperitoneally on days 1 and 4.

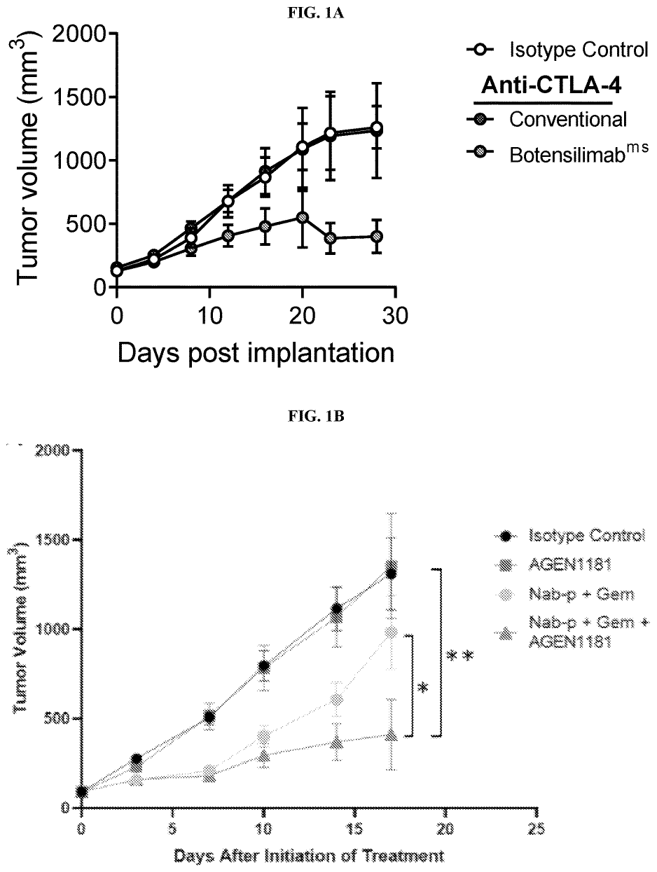

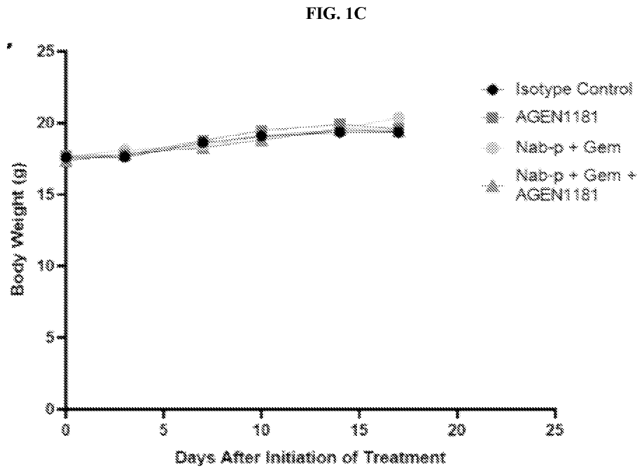

The results shown in FIG. 1A demonstrate that botensilimab, an Fc-engineered antibody has greater anti-tumor activity in a mouse model of pancreatic cancer compared to a conventional anti-CTLA-4 antibody that has a wild-type Fc. The results shown in FIG. 1B demonstrate that the tumors in mice treated with a combination of botensilimab and nab-paclitaxel+gemcitabine grew significantly slower than in mice treated with either botensilimab^{ms} alone (P value <0.0001) or nab-paclitaxel+gemcitabine (P value=0.0059). Further, the results shown in FIG. 1C demonstrate that there is no significant difference in body weight changes among the treatment groups, which indicates that there was no significant toxicity associated with these treatments.

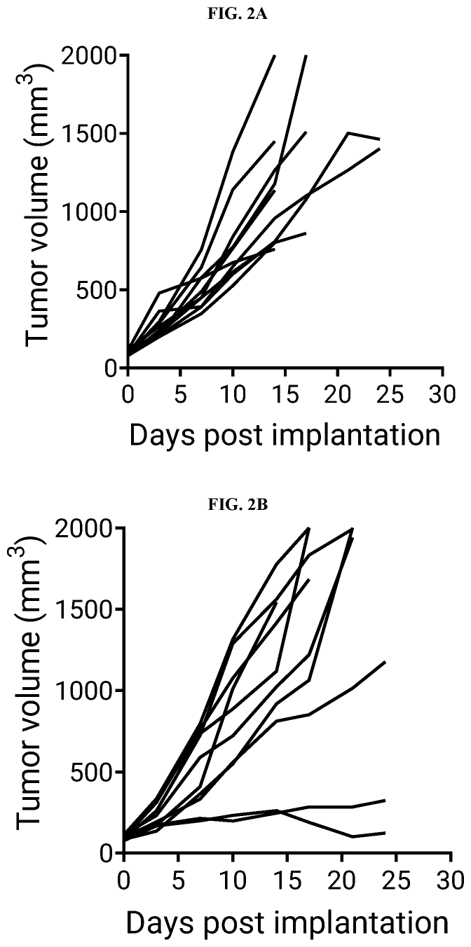

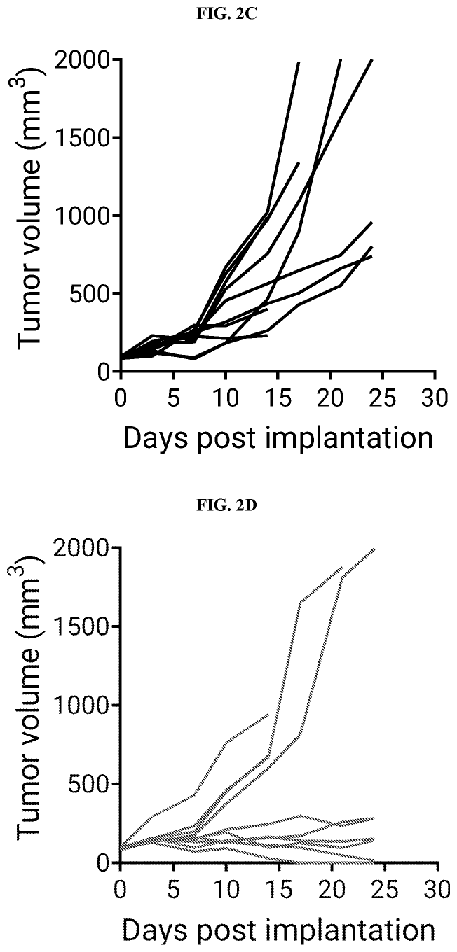

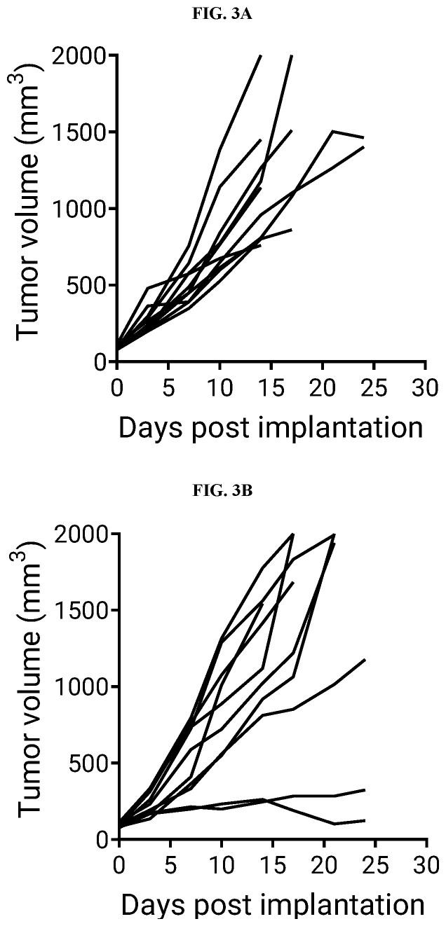

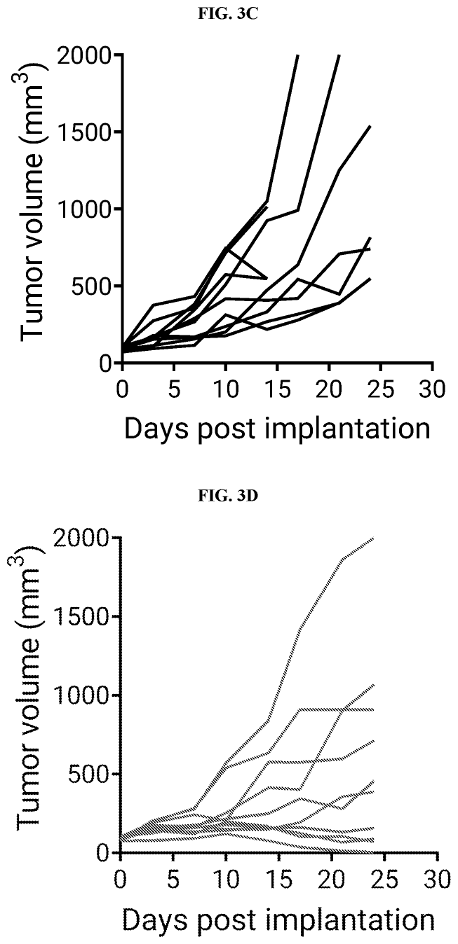

The results of FIGS. 2 and 3 show that botensilimab in combination with doublet chemotherapies show increased anti-tumor activity compared with the doublet chemotherapies alone. For the analysis of botensilimab in combination with the nab-paclitaxel+gemcitabine doublet, the individual tumor growth curves are shown in FIG. 2A (isotype control), FIG. 2B (botensilimab^{ms} monotherapy), FIG. 2C (nab-paclitaxel+gemcitabine), and FIG. 2D (botensilimab and nab-paclitaxel+gemcitabine). These individual treatment group results demonstrate that the majority of the mice treated with the combination of botensilimab and nab-paclitaxel+gemcitabine had tumor shrinkage with even complete remissions (3 out 10 mice). For the analysis of botensilimab in combination with the cisplatin+gemcitabine doublet, the individual tumor growth curves are shown in FIG. 3A (isotype control), FIG. 3B (botensilimab^{ms} monotherapy), FIG. 3C (cisplatin+gemcitabine), and FIG. 3D (botensilimab and cisplatin+gemcitabine).

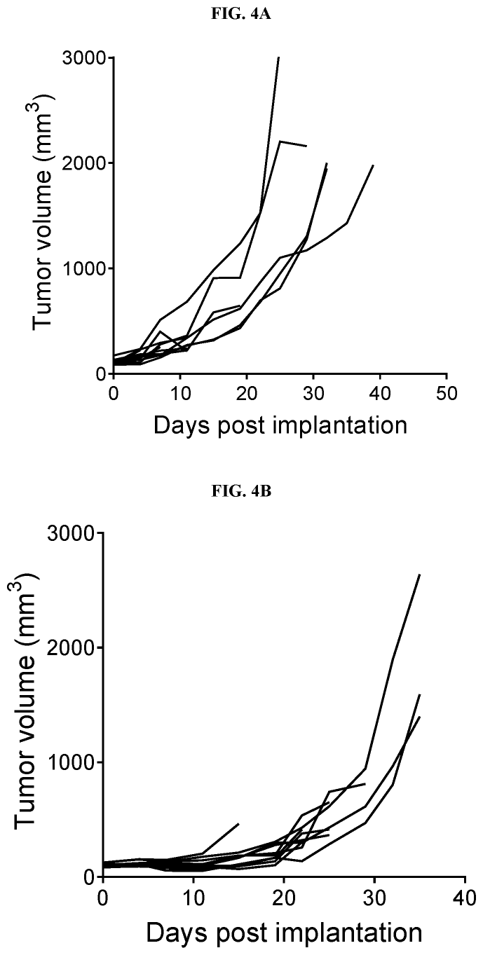

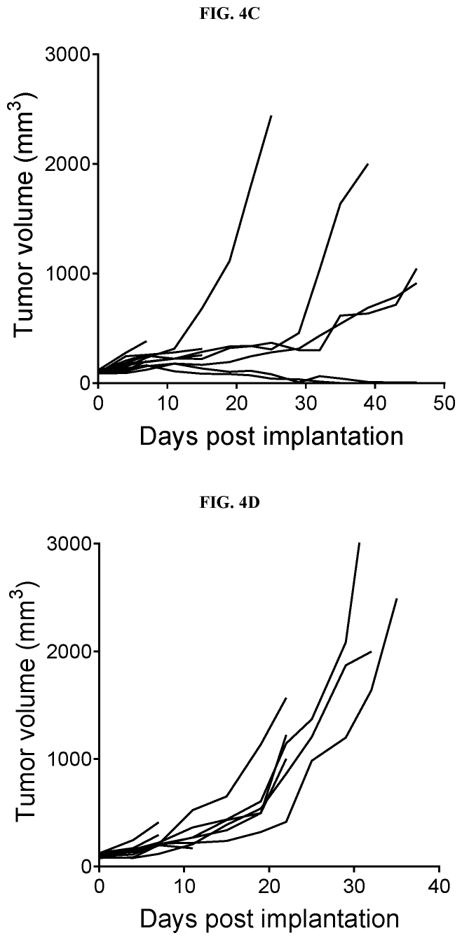

Further, the results shown in FIG. 4 demonstrate that botensilimab has greater anti-tumor activity in mice compared to an anti-PD-1 antibody. Individual tumor growth curves are shown in FIG. 4A (isotype control), FIG. 4B (triplet chemotherapy, Abraxane+cisplatin+gemcitabine), FIG. 4C (botensilimab^{ms} monotherapy), and FIG. 4D (anti-PD-1 antibody monotherapy).

These results demonstrate that botensilimab has promising preclinical activity in combination with nab-paclitaxel and gemcitabine that is superior to chemotherapy alone, in the difficult to treat KPC syngeneic mouse model for pancreatic ductal adenocarcinoma.

### Example 2—Phase 2 Study of Botensilimab (AGEN1181) in Combination with Nab-Paclitaxel and Gemcitabine for the Treatment of Metastatic Pancreatic Cancer

This Phase 2 study further evaluated the safety and efficacy of botensilimab in patients with metastatic pancreatic cancer who have progressed on prior 5FU+leucovorin+irinotecan+oxaliplatin (FOLFIRINOX).

### A. Study Design

### Overall Design

This was a prospective, multicenter, randomized, open label, Phase 2 clinical trial of botensilimab in combination with nab-paclitaxel+gemcitabine. The trial was conducted in two parts—Part 1 of the clinical trial was a safety lead-in to establish the safety of the proposed combination of botensilimab with nab-paclitaxel+gemcitabine and determined the botensilimab dose for Part 2. Part 2 was a randomized, open label assessment of botensilimab (at the dose level determined in Part 1) in combination with nab-paclitaxel+gemcitabine, and nab-paclitaxel+gemcitabine alone.

The study enrolled patients with metastatic pancreatic ductal adenocarcinoma who have progressed on a prior FOLFIRINOX regimen for their advanced, metastatic disease. The investigator assessed and documented in the patient's source documents, as part of the screening process, disease progression on any version of FOLFIRINOX for metastatic disease. Baseline study procedures included computed tomography (CT), preferred, or magnetic resonance imaging (MRI), only if CT was contraindicated, of the chest, abdomen and pelvis, routine laboratory testing and tumor markers.

### Study Treatments

Part 1—Safety Lead-In: Safety of the proposed combination was established based on modified 3+3 dose escalation rules with 18 patients.

Three patients were enrolled and treated at a fixed dose of botensilimab (Dose Level 1:50 mg Q6W)+gemcitabine (1000 mg/m²)/nab-paclitaxel (125 mg/m²) per SoC (Table 4). Based on the initial safety signals noted in these first 3 dose-limiting toxicity (DLT) evaluable patients treated at Dose Level 1, the botensilimab dose was escalated, expanded, or reduced as follows:
If none of the first 3 DLT evaluable patients at 50 mg experienced a DLT within the DLT evaluation interval, then an additional 3 patients were enrolled at the escalated Dose Level 2: botensilimab 150 mg.
If 1 of the first 3 DLT evaluable patients at 50 mg experienced a DLT within the DLT evaluation interval, then an additional 3 patients were enrolled at this Dose Level 1: botensilimab 50 mg.
Dose de-escalation: If >1 of the first 3 or 6 DLT evaluable patients at 50 mg experienced a DLT within the DLT evaluation interval, then an additional 3 patients were enrolled at the reduced Dose Level-1: botensilimab 25 mg.
If >1 of the first 3 or 6 DLT evaluable patients at 25 mg experienced a DLT within the DLT evaluation interval, the study did not proceed to Part 2.
If no more than 1 of the 6 DLT evaluable patients experienced a DLT within the DLT evaluation interval for a particular dose level, then that dose level was considered tolerable for Part 2.

“DLT Evaluable” refers to any patient who received at least one dose of botensilimab and at least one cycle of gemcitabine/nab-paclitaxel or any patient who experienced a DLT was evaluable for toxicity.

The DLT Interval was from time of first administration of study treatment on cycle 1, day 1 through the end of Cycle 1 (Day 41).

Dose limiting toxicity (DLT) was defined by the occurrence of the following AE considered related to botensilimab (including possibly related) during the DLT interval, Cycle 1/Day 1 through the end of Cycle 1 (Day 41) as defined by CTCAE version 5.0.

DLT was defined by the occurrence of a Grade≥3 AE (as defined by CTCAE v5.0) related (likely or possibly) to botensilimab during the DLT interval (C1/D1 through the end of Cycle 1 [Day 41]), with the following exceptions: any Grade fatigue, any Grade alopecia, any Grade cytopenia, any Grade nausea/vomiting, Grade 3≥immune related adverse event (irAE) which resolved to ≤Grade 2 within 3 days with appropriate management, any Grade endocrinopathy that was adequately controlled by hormonal replacement, and any laboratory abnormalities that did not require intervention and were deemed clinically insignificant by the investigator.

Part 2—Randomization Phase: Following the completion of Part 1, a total of 60 patients were accrued with 30 randomized to each arm as detailed below, in order to meet 48 evaluable patients. The two arms included:
1. Arm A (Combination): Botensilimab (Dose as determined in Part 1 Q6W×3 doses)+gemcitabine (1000 mg/m²)/nab-paclitaxel (125 mg/m²) per SoC (Table 4)
2. Arm B (Standard of Care): gemcitabine (1000 mg/m²)/nab-paclitaxel (125 mg/m²) per SoC (Table 4).

The dose level of botensilimab selected for Part 2 was in part based upon all available safety data obtained from Part 1. In addition, all available clinical safety data as well as pharmacokinetic, pharmacodynamic, and efficacy data obtained from other applicable botensilimab studies were considered as part of dose selection decision making for Part 2.

During the treatment period, patients had routine clinical visits for administration of study treatment and monitoring of safety, well-being, and changes in disease status. CA 19-9 (or CA 125, or CEA if not expressers of CA 19-9) was collected at the beginning (Day 1) of each cycle and day 1 of each chemotherapy cycle.

Patients in Arm A received up to 3 doses of Botensilimab (3 cycles). Patients in Arms A and B continued on gemcitabine (1000 mg/m²)/nab-paclitaxel (125 mg/m²) per SoC (Table 4) until intolerable toxicity, disease progression, death, or patient washed to discontinue from study treatment for any reason.

Anti-tumor efficacy was determined via imaging assessments, which were performed within 21 days before randomization (the first dose of study drug for Part 1 patients). On-study tumor assessment occurred every 8 weeks (+/−7 days) from randomization for the first 24 weeks and then every 12 weeks (+/−7 days). Disease response was evaluated periodically by independent blinded central review, blinded to treatment assignment. Confirmation of progressive disease (PD) was demonstrated by imaging at 4 to 6 weeks (but not later) after progression has been diagnosed and reviewed by the central radiologist prior to discontinuation of study treatment.

Safety was assessed via monitoring of AE, SAE, treatment discontinuation due to AE, physical examinations, vital signs, hematology, and chemistry laboratories.

Patients who discontinued study treatment for reasons other than progressive disease (PD) continued with imaging assessments per the SoA, visited Q6W and imaging performed at Week 8, 16, 24 and Q12W after that until the start of additional anti-neoplastic therapy or disease progression.

For survival follow-up, patients who began additional anti-neoplastic therapy, or who have discontinued imaging on study for other reasons, were followed Q12W for survival status via telephone call until the end of the study.

TABLE 3
Study Treatments

| Study Treatment | | | |
|---|---|---|---|
| Name: | Botensilimab | Gemcitabine | Nab-paclitaxel |
| Dosage Formulation: | Solution for | Lyophilized | Injectable |
| | infusion | powder | suspension |
| Unit Dosage Strength: | 50 mg/mL | 200 mg or 1 g | 100 mg |
| Dosage Level: | 25 mg | 1000 mg/m² per | 125 mg/m² |
| | 50 mg | standard of care | per standard |
| | 150 mg | | of care |
| Route of | intravenous | intravenous | intravenous |
| Administration: | infusion | infusion | infusion |

TABLE 4
Standard of Care Dose Reduction

| Dose level | Nab-paclitaxel | Gemcitabine |
|---|---|---|
| Level 0 (baseline) | 125 mg/m² | 1000 mg/m² |
| Level 1 | 100 mg/m² | 800 mg/m² |
| Level 2 | 75 mg/m² | 600 mg/m² |
| If additional dose | Discontinued | Discontinued |
| reduction required | | |

Doses of nab-paclitaxel and gemcitabine were reduced in individual patients in accordance with the schedule in Table 4. In general, doses that were reduced for toxicity were not escalated back to the starting level. In some cases, growth factors were used to treat hematologic toxicity and did not constitute a dose reduction.

A maximum of a 4-week treatment delay in chemotherapy was permitted to allow recovery of toxicities or other concurrent illness that prevented the patient from receiving chemotherapy. In the event dose modifications were required at the beginning of a cycle or within a cycle due to hematologic toxicities, doses of nab-paclitaxel and gemcitabine were adjusted. During Cycle 1, if the patient experienced a Grade 4 hematologic AE related to chemotherapy as determined by the investigator, dose was reduced to Dose Level-1 for Cycle 2 per Table 4.

In the event that patients had treatment delayed within a treatment cycle due to hematologic toxicities, those doses held during a cycle were not made up. Dose modifications due to hematologic toxicity (as represented by the blood counts and toxicities, below) within a treatment cycle were adjusted. If patients did not experience resolution of neutropenia within 28 days, despite uninterrupted G-CSF treatment, study treatment was discontinued. Febrile patients (regardless of neutrophil count) had their chemotherapy treatment interrupted. A full sepsis diagnostic work-up was performed while continuing broad spectrum antibiotics. If cultures were positive, the antibiotic was changed, depending on the sensitivity profile of the isolated organism. Patients with persistent fever after 3 weeks, despite uninterrupted antibiotic treatment, discontinued study treatment. Febrile neutropenic patients also received G-CSF, in addition to antibiotic treatment, to hasten the resolution of their febrile neutropenia (following current institutional guidelines). In all cases, blood counts returned to baseline levels before resuming chemotherapy treatment.

Doses were reduced for non-hematologic toxicities that occurred despite adequate background medical therapy. If the toxicity only affected neuropathy, then only nab-paclitaxel was reduced. Nab-paclitaxel treatment was withheld in patients who experienced≥Grade 3 peripheral neuropathy. Gemcitabine administration continued during this period. Nab-paclitaxel treatment was resumed at the next lower dose level as per Table 4 in subsequent cycles after the peripheral neuropathy improved to ≤Grade 1. The time to resolution to Grade≤1 was the adverse event duration used for adverse event reporting. Patients that experienced peripheral neuropathy that required a delay in scheduled nab-paclitaxel dosing for ≥28 days discontinued nab-paclitaxel.

Patients who developed Grade 2 or 3 cutaneous toxicity had their nab-paclitaxel and gemcitabine dose reduced to the next lower dose level as per Table 4. If the patient continued to experience these reactions, despite dose reduction, treatment was discontinued. Patients who developed Grade 4 cutaneous toxicity had study treatment discontinued.

If Grade 2 mucositis or diarrhea occurred, nab-paclitaxel and gemcitabine was withheld until resolution to ≤Grade 1, then reinstituted at the next lower dose level as per Table 4. Patients who developed Grade 3 mucositis or diarrhea had study treatment discontinued.

Asymptomatic or clinically mild pulmonary embolism was treated with low-molecular weight heparin without interruption of therapy. Moderate to severe pulmonary embolism required permanent discontinuation of chemotherapy. Pulmonary toxicity has been reported for both gemcitabine and paclitaxel. Combination chemotherapy of gemcitabine and paclitaxel has shown a higher incidence of pneumonitis (4%) compared to either drug alone. Early detection and treatment was required as it may be life-threatening or even fatal.

During study participation, patients were carefully monitored for signs and symptoms of pneumonitis (i.e., episodes of transient or repeated dyspnea with unproductive persistent cough or fever) and, if observed, immediate clinical evaluation and timely institution of appropriate management was performed (emphasizing the need for corticosteroids if an infectious process has been ruled out as well as appropriate ventilation and oxygen support when required). If asymptomatic Grade 1 pneumonitis seen only on scans was noted, a patient continued with gemcitabine and nab-paclitaxel provided they received clinical benefit from the study and after a thorough discussion with the medical monitor. Administration of study treatment was permanently discontinued upon diagnosis of >Grade 1 interstitial pneumonitis.

Study drug administration was interrupted upon diagnosis of >Grade 1 interstitial pneumonitis and patients permanently discontinued from further study drug treatment. After ruling out an infectious etiology, intravenous high-dose corticosteroid therapy and secondary pathogen coverage was instituted without delay. Patients with an added immunological component required immune modulation with azathioprine or cyclophosphamide. Appropriate ventilation and oxygen support was used when required.

Granulocyte colony-stimulating factor (G-CSF) was given according to institutional guidelines. If no institutional guidelines, G-CSF was given per the ASCO Practice Guideline Recommendations for the Use of White Blood Cell Growth Factors: Guideline Summary Appendix C.

Adverse events (AEs) associated with botensilimab exposure may have had an immunologic etiology. In some cases, these immune-related AEs (irAEs) occurred shortly after the first dose or several months after the last dose of treatment and affected more than one body system simultaneously. Early recognition of irAE and initiation of appropriate immunosuppressive treatment was critical to reduce complications and improve patient outcomes. Based on existing clinical trial data, most irAEs were reversible and managed with interruptions of botensilimab, administration of corticosteroids and/or infliximab, and/or other supportive care.

### Study Drug Administration

An individual cycle of therapy was defined as 6 weeks, with administration of botensilimab on Day 1 of each cycle (Arm A), and administration of gemcitabine/nab-paclitaxel on Days 1, 8 and 15 Q6W. Patients received 3 doses of botensilimab and continued on gemcitabine/nab-paclitaxel until disease progression, unacceptable toxicity, or patient wished to discontinue from study treatment for any reason.

The sequence of drug administration was botensilimab, nab-paclitaxel, then gemcitabine. Nab-paclitaxel was given before gemcitabine because it may be taken up by the process of macropinocytosis. Gemcitabine was given next because albumin-bound paclitaxel potentially decreases cytidine deaminase which potentiates gemcitabine activity (less degradation of gemcitabine by the enzyme).

Supportive medications included: Palonosetron (Aloxi®) 0.25 mg IV, fosaprepitant (Emend®) 150 mg IV and dexamethasone 12 mg IV, or equivalent antiemetic regimen within 30 minutes prior to chemotherapy. Patients continued oral antiemetic prophylaxis at home for 2 days after chemotherapy. The type of antiemetic prophylaxis used varied based on institutional procedures, but an effort was made to minimize corticosteroid dose and duration, i.e., did not use dexamethasone on days 2 and 3 unless needed, alternatives included ondansetron if palonosetron was not given or olanzapine.

Botensilimab was administered via IV infusion over 30 (+5) minutes. Patients were observed for 30 minutes after the end of the infusion for infusion-related reactions. Measurement of vital signs occurred prior to the start of each infusion and at the end of each infusion for all cycles. Infusions were followed immediately with a saline flush of the IV line, per institutional guidelines.

Gemcitabine/nab-paclitaxel was administered per Table 4, and according to the gemcitabine and nab-paclitaxel prescribing information.

### Number Of Patients

Part 1 had 18 patients. In Part 2, approximately 60 eligible patients (30 patients randomized to each arm) were enrolled.

In Part 1, patients who failed to meet the criteria to be considered DLT evaluable were replaced. For Part 2 of the study, patients that received at least one dose of study treatment was not replaced.

Patients that met eligibility to be enrolled but later determined ineligible on C1/D1 did not receive study treatment and were replaced.

### Treatment Beyond Disease Progression

Confirmatory imaging was performed at 4-6 weeks for patients with progressive disease (PD) who remained on study treatment per protocol. If PD was confirmed at the confirmatory scan by a 10% increase or greater for the sum of measurable lesion(s), unequivocal progression of non-measurable lesion(s), or the appearance of a new lesion, the patient was discontinued from treatment.

Patients who tolerated the drug and were considered by the investigator to be deriving clinical benefit were permitted, with the Sponsor's approval, to continue with treatment beyond initial RECIST 1.1-defined PD if they met the following clinical stability criteria:
Absence of clinical symptoms and signs (including worsening of laboratory values) indicating PD.
No decline in ECOG status (Appendix A) attributed to underlying malignancy
The patient tolerated study drug; and continued treatment will not prevent or delay treatment that may prevent serious complications of disease.

### Treatment Duration

Part 1: Patients received up to 3 doses of botensilimab (3 cycles) at the assigned dose and in combination with gemcitabine (1000 mg/m²)/nab-paclitaxel (125 mg/m²) per SoC (Table 4). After 3 doses of botensilimab, patients continued the gemcitabine/nab-paclitaxel per SoC until disease progression, unacceptable toxicity, or patient wished to discontinue from study treatment for any reason.

Part 2: Patients in Arm A (Combination) received up to 3 doses of botensilimab (3 cycles) at the dose determined in Part 1 in combination with gemcitabine (1000 mg/m²)/nab-paclitaxel (125 mg/m²) per SoC (Table 4). After 3 doses of botensilimab, patients continued the gemcitabine/nab-paclitaxel per SoC until disease progression, unacceptable toxicity, or patient wished to discontinue from study treatment for any reason.

Patients in Arm B (SoC) were treated with gemcitabine (1000 mg/m²)/nab-paclitaxel (125 mg/m²) per SoC (Table 4) until disease progression, unacceptable toxicity, or patient wished to discontinue from study treatment for any reason. After treatment discontinuation, patient was followed for safety at 30 (+7) days and 90 (+7) days, and for long-term follow-up every 3 months for 12 months from last administration.

If the patient remained eligible to remain on the study despite study treatment discontinuation (e.g., due to toxicity), they continued with scheduled imaging assessments and concurrent office visits.

If the study was not terminated for a reason provided in Section 9.4, the overall trial ended approximately 12 months after the last randomized patient completed the last dose of botensilimab, withdrew from the trial or was lost to follow-up (i.e., the patient was unable to be contacted by the Investigator).

### Prohibited Medications and Treatments

Medications or vaccinations specifically prohibited in the exclusion criteria were not allowed during the study. If there was a clinical indication for any medication or vaccination specifically prohibited during the study, discontinuation from study therapy or vaccination was required. The final decision on any supportive therapy or vaccination rested with the Investigator and/or the patient's primary physician. However, the decision to continue the patient on study treatment required the mutual agreement of the Investigator, the Sponsor, and the patient.

Listed below are specific restrictions for concomitant therapy instituted during the course of the study:
Antineoplastic systemic chemotherapy or biological therapy other than SoC.
Immuno-oncology therapies not specified in this protocol.
Investigational agents other than the assigned study treatment.
Systemic glucocorticoids (>10 mg prednisone equivalent for >1 week) for any purpose other than to treat an immune-related adverse event. Note: Use of prophylactic corticosteroids to avoid allergic reactions (e.g., IV contrast dye or transfusions) was permitted, as was the use of inhaled steroids or intranasal or local injection of corticosteroids.
The metabolism of paclitaxel is catalyzed by CYP2C8 and CYP3A4. Caution was exercised when administering nab-paclitaxel concomitantly with medicines known to inhibit or induce either CYP2C8 or CYP3A4.

Patients treated with any prohibited medication (excluding the exceptions noted above) for clinical management were removed from the study. All treatments that the Investigator considered necessary for a patient's welfare were administered at the discretion of the Investigator in keeping with the community standards of medical care. All concomitant medications were recorded on the electronic case report form (eCRF) including all prescription, over-the-counter products, herbal supplements, and IV medications and fluids. If changes occurred during the study period, documentation of drug dosage, frequency, route, and date was noted in the eCRF.

### Surgery and Radiation Therapy

If a patient required surgery for management of progressive malignant disease, they were discontinued from study treatment. In the instance of surgery for bowel obstruction, if there was no confirmed PD, the patient continued receiving treatment on study. Palliative radiotherapy on non-target lesions was permitted following discussion with the Sponsor based on the specific clinical scenario.

### Permanent Discontinuation Of Study Drug Treatment

Botensilimab was permanently discontinued for any of the following reasons:
Occurrence of an immune-related adverse event that met the criteria for discontinuation.
Confirmed PD unless the patient was considered by the Investigator to derive clinical benefit from the treatment, the patient was clinically stable, and there was approval from the Sponsor.
Clinical progression, in absence of radiologic progression on the basis of RECIST 1.1 as suggested by one or more of the following:
   Signs and/or symptoms consistent with clinically significant progression of disease, including worsening of laboratory values, appearance of new lesion(s)/worsening of the lesion best seen clinically, etc.
Two or more consecutive doses of study therapy were missed due to non-compliance, unless otherwise approved by Sponsor.

Tumor flare phenomenon, defined as local pain, irritation, or rash localized at sites of known or suspected tumor, did not require treatment discontinuation.

### Definition Of End Of Study

If the study was not terminated for a reason provided above, the overall study ended 24 months after the last patient completed the 90-day safety follow-up visit; if the last patient withdrew from the study, or was lost to follow-up (i.e., the patient was unable to be contacted by the Investigator) prior to the 90-day safety follow-up visit, the study ended approximately 24 months from the date of that event.

### B. Study Population

### Inclusion Criteria

In order to participate in the study, a patient must meet all of the following inclusion criteria:
1. Voluntarily agree to participate by giving signed, dated, and written informed consent prior to any study-specific procedures.
2. ≥18 years of age.
3. Histologically confirmed diagnosis of pancreatic ductal adenocarcinoma.
4. Must have had disease progression on any version of FOLFIRINOX for metastatic disease.
5. Eastern Cooperative Oncology Group (ECOG) performance status of 0 or 1.
6. Life expectancy of at least 3 months.
7. Measurable disease on baseline imaging per RECIST 1.1 criteria.
8. <Grade 2 pre-existing peripheral neuropathy per NCI CTCAE, Version 5.0.
9. Acceptable coagulation status as indicated by an INR≤1.5 times institutional upper limit of normal (ULN), except patients on anticoagulation who can be included at the discretion of the investigator.
10. Adequate organ function defined as the following laboratory values within 7 days prior to first dose of study drugs, except where noted below:
   a. Neutrophils >1500/μL (stable off any growth factor within 4 weeks prior to first dose of study drugs).
   b. Platelets>100×10³/μL (transfusion to achieve this level is not permitted within 2 weeks prior to first dose of study drugs).
   c. Hemoglobin>9.0 g/dL (transfusion to achieve this level is not permitted within 2 weeks prior to first dose of study drugs).
   d. Creatinine clearance ≥30 mL/min (measured or calculated per institutional standards).
   e. Aspartate aminotransferase (AST)/alanine aminotransferase (ALT)<3.0×ULN.
   f. Direct bilirubin <1.5×ULN (except patients with Gilbert syndrome who must have a total bilirubin level of <3.0×ULN).
   g. Serum albumin≥3.0 g/dL (must be confirmed within 3 days of prior to first dose of study drugs).
11. Female patients of childbearing potential (WOCBP) must have a negative urine or serum pregnancy test at screening (within 72 hours of first dose of study drugs). WOCBP must agree to use highly effective contraceptive measures starting with the screening visit through 6 months after the last dose of study drug(s). Non-childbearing potential is defined as:
   a. ≥50 years of age and has not had menses for greater than 1 year.
   b. Amenorrheic for ≥2 years without a hysterectomy and bilateral oophorectomy and a follicle-stimulating hormone value in the postmenopausal range upon pre-study (screening) evaluation.
   c. Status is post hysterectomy, bilateral oophorectomy, or tubal ligation.
12. Male patients with a female partner(s) of childbearing potential must agree to use highly effective contraceptive measures throughout the study starting with the screening visit through 90 days after the last dose of study drug(s) is received. Males with pregnant partners must agree to use a condom; no additional method of contraception is required for the pregnant partner.
13. Willing and able to comply with the requirements of the protocol.

### Exclusion Criteria

Participants were excluded from the trial if any of the following criteria applied:
1. Received more than one prior regimen (i.e., FOLFIRINOX) for their metastatic disease.
2. History of central nervous system (CNS) metastasis.
3. Concurrent malignancy (present during screening) requiring treatment or history of prior malignancy active within 2 years prior to the first dose of study drugs (i.e., patients with a history of prior malignancy were eligible if treatment was completed at least 2 years prior to first dose of study drugs and the patient has no evidence of disease). Patients with history of prior early-stage basal/squamous cell skin cancer or noninvasive or in situ cancers who have undergone definitive treatment at any time were also eligible.
4. Uncontrolled intercurrent illness, including but not limited to clinically significant (i.e., active) cardiovascular disease: cerebral vascular accident/stroke or myocardial infarction within 6 months of enrollment, unstable angina, congestive heart failure (New York Heart Association class≥III), or serious uncontrolled cardiac arrhythmia requiring medication.
5. Active, uncontrolled infections, requiring systemic intravenous anti-infective treatment within 2 weeks prior to first dose of study drugs.
6. Major surgery within 4 weeks prior to signing of informed consent form (ICF).
7. Prior treatment with an immune checkpoint inhibitor.
8. Refractory ascites defined as requiring 2 or more therapeutic paracenteses within the last 4 weeks or ≥4 within the last 90 days or ≥1 time within the last 2 weeks prior to signing of ICF or requiring diuretics within 2 weeks of signing of ICF.
9. Partial or complete bowel obstruction within the last 3 months prior to signing ICF, signs/symptoms of bowel obstruction, or known radiologic evidence of impending obstruction.
10. Clinically significant gastrointestinal (GI) disorders including:
   a. GI perforation or unhealed ulcerations<6 months prior to signing of ICF. Patients must have documented evidence (e.g., upper endoscopy, colonoscopy) of completely healed area of prior perforation or ulceration.
   b. Clinically significant GI bleeding<3 months prior to signing of ICF.
   c. History of active Crohn's disease or ulcerative colitis.
11. Treatment with one of the following classes of drugs within the delineated time window prior to first dose of study drugs:
   a. Cytotoxic agent within 3 weeks.
   b. Monoclonal antibodies, antibody-drug conjugates, radioimmunoconjugates, or investigational drug, within 4 weeks, or 5 half-lives, whichever is shorter.
   c. Small molecule/tyrosine kinase inhibitors within 14 days.
12. Previous severe acute respiratory syndrome coronavirus 2 (SARS-COV-2) infection within 10 days for mild or asymptomatic infections or 20 days for severe/critical illness prior to first dose of study drugs.
13. SARS-COV-2 vaccine<7 days prior to first dose of study drugs.
14. Known allergy or hypersensitivity to any of the study drugs or any of the study drug excipients.
15. Symptomatic interstitial lung disease (ILD), history of ILD or any lung disease which may interfere with detection and management of new immune-related pulmonary toxicity.
16. History of allogeneic organ transplant.
17. Psychiatric or substance abuse disorders that would interfere with cooperation with the requirements of the study.
18. Patients with a condition requiring systemic treatment with either corticosteroids (>10 mg daily prednisone equivalent) within 14 days or another immunosuppressive medication within 30 days prior to first dose of study drugs. Inhaled or topical steroids, and adrenal replacement steroid doses (≤10 mg daily prednisone equivalent), are permitted in the absence of active autoimmune disease.
19. Active autoimmune disease or history of autoimmune disease that required systemic treatment within 2 years prior to first dose of study drugs (i.e., with use of disease-modifying agents or immunosuppressive drugs).
20. Pregnant or breastfeeding patients.
21. Uncontrolled infection with HIV. Patients on stable highly active antiretroviral therapy (HAART) therapy with undetectable viral load and normal CD4 counts for at least 6 months prior to signing of ICF. Serological testing for HIV at screening was not required.
22. Known to be positive for hepatitis B (HBV) surface antigen, or any other positive test for HBV indicating acute or chronic infection. Patients who are or have received anti-HBV therapy and have undetectable HBV DNA for at least 6 months prior to signing of ICF. Serological testing for HBV at screening was not required.
23. Known active hepatitis C (HCV) as determined by positive serology and confirmed by polymerase chain reaction (PCR). Patients on or having received antiretroviral therapy are eligible provided they are virus-free by PCR for at least 6 months prior to signing of ICF. Serological testing for HCV at screening was not required.
24. Dependence on total parenteral nutrition.
25. Patients with concurrent diarrhea>grade 1 at time of randomization despite optimal treatment with SoC pancreatic enzymes.
26. Known active or latent tuberculosis (testing at screening not required).
27. Any condition in the opinion of the principal investigator that might interfere with the patient's participation in the study or in the evaluation of the study results.
28. Unwillingness or inability to comply with procedures required in this protocol.

### C. Study Assessments and Procedures

All screening evaluations were completed and reviewed to confirm that potential patients met all eligibility criteria. The Investigator maintained a screening log to record details of all patients screened and to confirm eligibility or record reasons for screening failure, as applicable.

Procedures conducted as part of the patient's routine clinical management (e.g., blood count) and obtained before signing of the informed consent form (ICF) were utilized for screening or baseline purposes provided the procedure met the protocol-specified criteria and were performed within the time frame of other baseline assessments.

Repeat or unscheduled samples and images were taken for safety reasons or for technical issues with the samples.

### Screening

The Investigator, or qualified designee, obtained documented informed consent from each potential patient prior to participating in the clinical study. After a patient signed an ICF, the patient was assigned a unique, sequential patient number. Once a number was assigned, it cannot be reassigned if the original patient was found to be ineligible or withdrew consent.

Patients who fulfilled all the inclusion criteria and none of the exclusion criteria were enrolled into the study. Patients who did not meet the inclusion and exclusion criteria were considered screen failures, and their demographic information and reason for screen failure was documented.

During the screening period, attention was given to washout periods for prior treatments and prohibited medications.

### Treatment and Evaluation Period

Patients continued to receive all assessments while they were actively receiving treatment.

### Treatment Discontinuation Visit

The discontinuation visit (≤7 days after the last dose) occurred at the time study treatment was discontinued for any reason. If the discontinuation visit occurred approximately 30 days from the last dose of study treatment, at the time of the mandatory 30-Day Safety Follow-Up Visit, procedures were not repeated.

### 30-Day Safety Follow-up Visit

The mandatory 30-Day Safety Follow-up Visit was conducted for all patients 30 days (±7 days) after the last dose of study treatment or before initiation of a new antineoplastic treatment, whichever came first. Patients with an AE of Grade≥1 were further followed until the resolution of the AE to Grade 0 to 1 or until initiation of a new antineoplastic therapy, whichever occurred first. Patients with a TRAE ongoing at the safety visit were followed until the TRAE resolved, becomes stable, or was considered not clinically significant by the Investigator.

All patients who discontinued treatment for any reason will also have a 90-Day Safety Follow-up Visit (±7 days).

### Efficacy Follow-Up (Off Treatment, on Study)

Patients who discontinued study treatment for reasons other than PD continued with visits and imaging assessments on schedule.

### Survival Follow-Up

For patients who were no longer continuing with study visits and imaging (e.g., after starting subsequent anti-neoplastic therapy), the patient was contacted by telephone Q12W (±14 days) to assess survival status until the overall study ended. Survival data was obtained from public records for patients who have been lost to follow-up.

### Post Study Anti-cancer Therapy

The Investigator, or qualified designee, reviewed all new anti-cancer therapy initiated after the last dose of study drug. If a patient initiated a new anti-cancer therapy within 4 weeks after the last dose of study drug, the 30-Day Safety Follow-up Visit occurred before the first dose of the new therapy. Once new anti-cancer therapy was initiated, patients moved into Survival Follow-up.

### Efficacy Assessments

Response assessment was performed according to RECIST 1.1 (Eisenhauer 2009). For all patients, tumor response assessment was obtained by radiographic imaging with CT (preferred) or MRI (if CT is contraindicated) evaluation of the C/A/P (plus other regions as required for specific tumor types or disease history). In general, lesions detected at baseline were followed using the same imaging methodology and preferably the same imaging equipment at subsequent tumor evaluation visits.

Following initial objective response (PR or CR per RECIST 1.1) a confirmatory set of imaging studies was obtained 4 to 6 weeks later. Before stopping treatment, confirmation of PD was demonstrated by imaging at 4 to 6 weeks (but not later) after progression was diagnosed and reviewed by the central radiologist prior to discontinuation of study treatment.

Tumor responses to treatment were assigned based on evaluation of response of target, non-target, and new lesions according to RECIST 1.1 (all measurements were recorded in metric notation). To assess objective response, tumor burden at baseline wasestimated and used for comparison with subsequent measurements. At baseline, tumor lesions werecategorized in target and non-target lesions. Results for these evaluations were recorded with as much specificity as possible so that pre- and post-treatment results provided the best opportunity for accurately evaluating tumor response.

If a patient discontinued treatment but remained on study, scans were taken according to the respective schedule of assessment. Additional imaging was performed if clinically indicated per the Investigator's discretion.

### Tumor Imaging

Initial tumor imaging was performed during the screening period to establish a baseline of disease burden. Scans performed as part of routine clinical management were acceptable for use as a screening scan if they were of adequate quality and ≤21 days prior to the first dose.

The Investigator performed scans in addition to a scheduled study scan if clinically indicated per the Investigator's discretion. The timing of on-study imaging followed calendar days and was not adjusted for delays in treatment administration or for visits. The same imaging technique was used in a patient throughout the study for consistency.

### Brain Imaging

MRI, with and without contrast, was the preferred brain imaging modality; however, CT was acceptable if MRI was clinically contraindicated. Patients with a history of CNS metastases received brain imaging on the same schedule as the chest/abdomen/pelvis imaging.

During the study, brain CT/MRI scans were conducted if clinically indicated by development of new symptoms.

### Assessment Of Safety

The safety profile of the study treatment was assessed through the recording, reporting, and analyzing of baseline medical conditions, AEs, physical examination findings, including vital signs, and laboratory tests. Comprehensive assessment of any apparent toxicity experienced by the patient was performed throughout the course of the study, from the time of the patient's signature of informed consent. Study site personnel reported any AE, whether observed by the Investigator or reported by the patient

### Primary Efficacy Analyses

PFS was defined as time from randomization until progression assessed by IRC per RECIST 1.1 or death, whichever came first. Patients without an event (death or PD) at the analysis cutoff date were censored on the date of last tumor assessment or start of new anti-cancer therapy. The median PFS and its 95% CI, if estimated, were constructed with generalized Brookmeyer and Crowley method (Brookmeyer 1982). The cumulative probability of PFS at 3-month intervals was calculated using Kaplan-Meier method for each treatment arm and presented with a two-sided 95% CI using Greenwood's formula. The PFS censoring rule followed the FDA Guidance for Industry Clinical Trial Endpoints for the Approval of Cancer Drugs and Biologics (Food and Drug Administration 2007). Data for patients without disease progression or death at the time of analysis was censored at the time of the last adequate tumor assessment. Data for patients who were lost to follow-up prior to documented disease progression were censored at the last adequate tumor assessment date when the patient was known to be progression free. Data for patients who started to receive new anti-cancer therapy was censored at the last adequate tumor assessment date prior to the introduction of new therapy.

At final analysis, PFS distribution between Arms A and B were compared descriptively in a log-rank test. The hazard ratio between arms and its 2-sided 95% CI wasestimated from the cox regression model. More details of PFS analysis and censoring were provided in the SAP.

The final analysis, which was the primary analysis of the study, was performed after approximately 46 PFS events have been observed per IRC review or 12 months after the last patient was randomized, whichever occurred earlier. Additional analysis included updated efficacy and safety data was performed upon completion of the study.

### Secondary Efficacy Analyses

### Objective Response Rate (ORR)

ORR was defined as the proportion of patients who had best overall response (BOR) of objective responses (CR or PR). BOR was defined as the best response recorded from randomization until data cutoff, disease progression, or the start of new anti-cancer treatment. Patients with no post-baseline response assessment were considered as non-responders for BOR. Confirmed ORR assessed by IRC per RECIST 1.1 was summarized using the same methods as described in the CRR.

### Duration of Response (DOR)

DOR was defined as the time from initial objective response until the first documentation of progression assessed by IRC per RECIST 1.1 or death, whichever came first. DOR was summarized using the Kaplan-Meier method in the responders only. All the censoring rules for PFS analysis were applied to DOR as well. The cumulative probability of DOR at 3-month intervals was calculated and presented with a two-sided 95% CI.

### Overall Survival (OS)

OS, defined as time to death of any cause, was analyzed in the ITT Analysis Set; patients were censored either at the date that the patient was last known to be alive or the date of data cutoff, whichever came earlier. The median OS and cumulative probability of OS estimated at 6-month intervals was calculated using Kaplan-Meier estimates for each treatment arm and presented with a two-sided 95% CI. Descriptive comparison of OS between arms was made similarly as in PFS at the final analysis.

### Complete Response (CR)

Complete response was assessed by IRC per RECIST v1.1 criteria and CA 19-9 (or CA 125, or CEA if not expressers of CA 19-9) down to normal limits (from at least >2×ULN) at the time of CR assessment by IRC.

Complete response rate (CRR) in each arm was summarized, as well as its corresponding Clopper-Pearson 95% CI. In addition, the ORR difference between arms was calculated with 95% CI constructed using Miettinen Nurminen method.

### Change in CA 19-9

Change in CA 19-9 was evaluated from the start of study treatment until progressive disease or death or date of last tumor assessment or start of new anti-cancer therapy. CA 19-9 normalization was defined as a value of CA 19-9 down to normal limits (from at least >2×ULN). CA 125 or CEA was evaluated for patients who were not expressors of CA 19-9.

### Safety Analyses

All safety endpoints were analyzed in the Safety Analysis Set, using actual treatment assignments.

### Extent of Exposure

Extent of exposure to each study drug was summarized descriptively as the number of doses received (number and percentage of patients), duration of exposure (days), cumulative total dose received per patient (mg), dose intensity, and relative dose intensity. The number (percentage) of patients requiring dose interruption, dose delays, and drug discontinuation due to AEs was summarized for each study drug. Frequency of the above dose adjustments and discontinuation was summarized by category.

Patient data listings were provided for all dosing records and for calculated summary statistics.

### Adverse Events (AEs)

All AEs were coded in MedDRA v25.0 or higher and graded by NCI CTCAE v5.0. AEs that had an onset date or a worsening in severity from baseline (pre-treatment) on or after the first dose of study drug and up to 90 days following discontinuation of the study treatment (i.e., the last dose of randomized treatment) or until the initiation of the first subsequent anti-cancer therapy (including radiotherapy, with the exception of palliative radiotherapy) following discontinuation of study treatment (whichever occurs first) were considered a treatment-emergent adverse event (TEAE) and included in summary tables. All AEs, treatment-emergent or not, were included in the listings.

The incidence of TEAEs was reported as the number (percentage) of patients with TEAEs by system, organ, class, and preferred term. The number (percentage) of patients with TEAEs was also summarized by relationship to the study drug. TRAEs included those AEs considered by the investigator to be related to a study drug or with missing assessment of the causal relationship.

SAEs, deaths, TEAEs with Grade≥3 severity, irAEs, TRAEs and TEAEs leading to treatment discontinuation, dose interruption, or dose delay were summarized

### D. Objectives and Endpoints

TABLE 5
Objectives and endpoints

| | |
|---|---|
| Objectives | Endpoints |

Primary

| | |
|---|---|
| To assess the progression-free | Progression Free Survival (PFS), |
| survival (PFS) Response Evaluation | defined as the time from the date of |
| Criteria in Solid Tumors (RECIST) | randomization to the date of the first |
| 1.1 as determined by independent | objectively documented tumor |
| central review committee (IRC) of | progression, assessed by investigators |
| botensilimab in combination with | per RECIST 1.1, or death whichever |
| gemcitabine and nab-paclitaxel | occurred first. |
| (Arm A) and gemcitabine and nab- | |
| paclitaxel (Arm B) in the Intent-to- | |
| Treat (ITT) Analysis set. | |

Secondary

| | |
|---|---|
| To assess the safety and tolerability | Adverse events (AEs), serious adverse |
| of botensilimab when administered | events (SAEs), and AEs leading to |
| in combination with gemcitabine | treatment discontinuation or death, and |
| and nab-paclitaxel (Arm A) or of | laboratory abnormalities. |
| gemcitabine and nab-paclitaxel | OS, defined as the time from |
| alone (Arm B) in the Safety | randomization until death due to any |
| Analysis Set. | cause. |
| To assess the overall survival (OS) | Complete response as defined by |
| of botensilimab in combination with | RECIST 1.1 criteria and CA 19-9 (or |
| gemcitabine and nab-paclitaxel | CA 125, or CEA if not expressers of CA |
| (Arm A) and gemcitabine and nab- | 19-9) down to normal limits (from at |
| paclitaxel (Arm B) in the ITT | least >2× ULN). |
| Analysis Set. | Overall response rate, defined as the |
| To assess complete responses and | proportion of patients whose best |
| overall response of botensilimab in | overall response was complete response |
| combination with gemcitabine and | or partial response per RECIST v1.1. |
| nab-paclitaxel (Arm A) and | DOR, defined as the time from initial |
| gemcitabine and nab-paclitaxel | objective radiographic response until |
| (Arm B) in the ITT Analysis Set. | disease progression or death, whichever |
| To assess duration of response | occurred first. |
| (DOR) of botensilimab in | Change in CA 19-9 (or CA 125, or CEA |
| combination with gemcitabine and | if not expressers of CA 19-9). |
| nab-paclitaxel and gemcitabine and | Rates of normalization of CA 19-9 (or |
| nab-paclitaxel in the ITT Analysis | CA 125, or CEA if not expressers of CA |
| Set. | 19-9). |
| To assess changes in and the rates of | |
| normalization of CA 19-9 (or CA | |
| 125, or CEA if not expressers of CA | |
| 19-9) of botensilimab in | |
| combination with gemcitabine and | |
| nab-paclitaxel and gemcitabine and | |
| nab-paclitaxel in the ITT Analysis | |
| Set. | |

The final analysis, which was the primary analysis of the study, was performed after approximately 46 PFS events were observed per IRC review or 12 months after the last patient was randomized, whichever occurred earlier. Additional analysis included updated efficacy and safety data was performed upon completion of the study.

### E. Initial Results

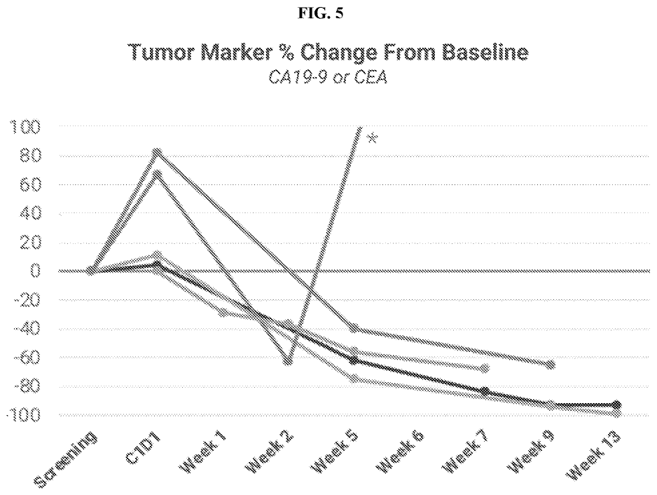

Initial analysis was performed on evaluable patients with metastatic pancreatic cancer, who have progressed on FOLFIRINOX, that received 150 mg botensilimab Q6W+gemcitabine (1000 mg/m²)/nab-paclitaxel (125 mg/m²) per SOC. All patients had liver metastasis. 80% of the evaluable patients (n=5) were found to experience sustained tumor marker reductions (FIG. 5). As shown in FIG. 5, four of the patients showed reduction in tumor marker (CA 19-9 or CEA) change from baseline. The fifth patient had clinical progression and was removed from the study. Two patients showed partial responses at 16 weeks with target lesion reduction of −47% and −37%, with both responses ongoing. Two other patients showed stable disease with tumor reduction of −20% and −13% at 8 weeks. Further analysis of the study results is ongoing.

### INCORPORATION BY REFERENCE

All patent and non-patent literature references cited above are incorporated herein by reference in their entirety.

1. A method of treating pancreatic cancer in a subject in need thereof, the method comprising administering to the subject an antibody that specifically binds to human Cytotoxic T-Lymphocyte Antigen 4 (CTLA-4) at a dose of 25 mg to 250 mg, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

2. A method of enhancing the activation of T cells in a subject who has pancreatic cancer, the method comprising administering to the subject an antibody that specifically binds to human CTLA-4 at a dose of 25 mg to 250 mg, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

3. The method of claim 1 or 2, wherein the antibody is administered at a dose of 25 mg to 200 mg.

4. The method of claim 1 or 2, wherein the antibody is administered at a dose of 50 mg to 200 mg.

5. The method of claim 1 or 2, wherein the antibody is administered at a dose of 25 mg, 50 mg, 75 mg, 100 mg, 125 mg, 150 mg, 175 mg, 200 mg, 225 mg, or 250 mg.

6. The method of any one of claims 1-5, wherein the antibody is administered intravenously.

7. The method of any one of claims 1-6, wherein the antibody is administered by intravenous infusion over about 30 minutes.

8. The method of any one of claims 1-7, wherein the antibody is administered once weekly.

9. The method of any one of claims 1-7, wherein the antibody is administered once every 2 weeks.

10. The method of any one of claims 1-7, wherein the antibody is administered once every 3 weeks.

11. The method of any one of claims 1-7, wherein the antibody is administered once every 4 weeks.

12. The method of any one of claims 1-7, wherein the antibody is administered once every 5 weeks.

13. The method of any one of claims 1-7, wherein the antibody is administered once every 6 weeks.

14. The method of any one of claims 1-7, wherein the antibody is administered intravenously at a dose of 25 mg once every 6 weeks.

15. The method of any one of claims 1-7, wherein the antibody is administered intravenously at a dose of 50 mg once every 6 weeks.

16. The method of any one of claims 1-7, wherein the antibody is administered intravenously at a dose of 75 mg once every 6 weeks.

17. The method of any one of claims 1-7, wherein the antibody is administered intravenously at a dose of 100 mg once every 6 weeks.

18. The method of any one of claims 1-7, wherein the antibody is administered intravenously at a dose of 150 mg once every 6 weeks.

19. The method of any one of claims 1-18, wherein the dose is a therapeutically effective amount.

20. The method of any one of claims 1-19, wherein the pancreatic cancer is pancreatic ductal adenocarcinoma.

21. The method of any one of claims 1-20, wherein the pancreatic cancer is unresectable.

22. The method of any one of claims 1-21, wherein the pancreatic cancer is metastatic.

23. The method of any one of claims 1-22, wherein the pancreatic cancer is relapsed and/or refractory.

24. The method of any one of claims 1-23, wherein the subject has received at least one prior chemotherapy.

25. The method of claim 24, wherein the at least one prior chemotherapy is 5-fluorouracil, leucovorin, irinotecan, or oxaliplatin.

26. The method of any one of the preceding claims, wherein the subject has previously been treated with 5-fluorouracil, leucovorin, irinotecan, and oxaliplatin.

27. The method of any one of the preceding claims, wherein the pancreatic cancer progressed following a standard of care treatment.

28. The method of any one of the preceding claims, wherein the method further comprises administering nab-paclitaxel to the subject.

29. The method of claim 28, wherein nab-paclitaxel is administered at a dose of 75 mg/m², 100 mg/m², or 125 mg/m².

30. The method of claim 28 or 29, wherein nab-paclitaxel is administered once every week.

31. The method of claim 28 or 29, wherein nab-paclitaxel is administered on days 1, 8, and 15 of a 6 week cycle.

32. The method of any one of the preceding claims, wherein the method further comprises administering gemcitabine to the subject.

33. The method of claim 32, wherein gemcitabine is administered at a dose of 600 mg/m², 800 mg/m², or 1000 mg/m².

34. The method of claim 32 or 33, wherein gemcitabine is administered once every week.

35. The method of claim 32 or 33, wherein gemcitabine is administered on days 1, 8, and 15 of a 6 week cycle.

36. The method of any one of claims 32-35, wherein the antibody is administered on day 1 of a 6 week cycle and nab-paclitaxel and gemcitabine are administered on days 1, 8, and 15 of the 6 week cycle.

37. The method of any one of claims 32-36, wherein the antibody is administered to the subject before nab-paclitaxel and gemcitabine.

38. The method of any one of claims 32-37, wherein nab-paclitaxel is administered before gemcitabine.

39. The method of any one of the preceding claims, wherein the cancer is refractory to a standard of care treatment.

40. The method of claim 39, wherein the standard of care treatment is chemotherapy or radiation.

41. The method of claim 39 or 40, wherein the standard of care treatment is 5-fluorouracil, leucovorin, irinotecan, and/or oxaliplatin.

42. The method of any one of the preceding claims, wherein the method reduces tumor size in the subject.

43. The method of any one of the preceding claims, wherein the method increases T-cell activation in the subject.

44. The method of any one of the preceding claims, wherein the method reduces the level of CA 19-9, CA 125, or CEA in the subject.

45. The method of any one of the preceding claims, wherein before administration of the antibody the subject has measurable disease on baseline imaging per RECIST 1.1.

46. The method of any one of the preceding claims, wherein before administration of the antibody the subject has an Eastern Cooperative Oncology Group performance status (PS) 0-1.

47. The method of any one of the preceding claims, wherein before administration of the antibody the subject has a predicted life expectancy of ≥12 weeks.

48. The method of any one of the preceding claims, wherein before administration of the antibody the subject has adequate organ function as defined by one or more of:
a) neutrophils≥1500/μL;
b) platelets≥100×10³/μL;
c) hemoglobin≥9.0 g/dL;
d) creatinine clearance ≥30 mL/min as measured or calculated per local institutional standards;
e) Aspartate aminotransferase (AST)/alanine aminotransferase (ALT)<3.0×upper limit of normal (ULN);
f) direct bilirubin <1.5×ULN (except patients with Gilbert syndrome who must have a total bilirubin level of <3.0×ULN); and/or
g) serum albumin ≥3.0 g/dL.

49. The method of any one of the preceding claims, wherein the subject does not have partial or complete bowel obstruction within the last 3 months, signs/symptoms of bowel obstruction, or known radiologic evidence of impending obstruction.

50. The method of any one of the preceding claims, wherein the subject has not received an immune checkpoint inhibitor therapy prior to administration of the antibody.

51. The method of any one of the preceding claims, wherein the subject has not received more than one chemotherapy regimen prior to administration of the antibody.

52. The method of any one of the preceding claims, wherein the subject does not have a history of central nervous system (CNS) metastases.

53. The method of any one of the preceding claims, wherein the subject does not have a concurrent malignancy that requires treatment or a history of prior malignancy that was active within 2 years prior to administration of the antibody.

54. The method of any one of the preceding claims, wherein the subject has not received a cytotoxic therapy or targeted therapy, within 3 weeks prior to administration of the antibody.

55. The method of any one of the preceding claims, wherein the subject has not received other monoclonal antibody therapy, antibody-drug conjugate therapy, or radioimmunoconjugate therapy, within 4 weeks prior to administration of the antibody.

56. The method of any one of the preceding claims, wherein the subject has not received small molecule tyrosine kinase inhibitor therapy within 2 weeks prior to administration of the antibody.

57. The method of any one of the preceding claims, wherein the subject does not have refractory ascites defined as requiring 2 or more therapeutic paracenteses within the last 4 weeks or ≥4 times within the last 90 days or ≥1 time within the last 2 weeks prior to administration of the antibody.

58. The method of any one of the preceding claims, wherein the subject does not have clinically significant cardiovascular disease.

59. The method of any one of the preceding claims, wherein the cancer has metastasized to the liver.

60. The method of any one of the preceding claims, wherein the antibody comprises the CDRH1, CDRH2, CDRH3, CDRL1, CDRL2, and CDRL3 amino acid sequences set forth in SEQ ID NOs: 1, 2, 3, 4, 5, and 6, respectively.

61. The method of any one of the preceding claims, wherein the antibody comprises a VH comprising the amino acid sequence set forth in SEQ ID NO: 7 and a VL comprising the amino acid sequence set forth in SEQ ID NO: 8.

62. The method of any one of the preceding claims, wherein the antibody comprises a human IgG1 heavy chain constant region comprising S239D/A330L/1332E mutations, numbered according to the EU numbering system.

63. The method of any one of the preceding claims, wherein the antibody comprises a heavy chain comprising the amino acid sequence set forth in SEQ ID NO: 9 and a light chain comprising the amino acid sequence set forth in SEQ ID NO: 10.

64. The method of any one of the preceding claims, wherein the antibody is botensilimab.

65. An antibody that specifically binds to human CTLA-4 for use in the treatment of pancreatic cancer, wherein the treatment is performed according to the method of any one of the previous claims.

66. An antibody that specifically binds to human CTLA-4 for use in the manufacture of a medicament for the treatment of pancreatic cancer, wherein the treatment is performed according to the method of any one of the previous claims.

67. Use of an antibody that specifically binds to human CTLA-4 for the treatment of pancreatic cancer, wherein the treatment is performed according to the method of any one of the previous claims.

68. Gemcitabine, nab-paclitaxel, and an antibody that specifically binds to human CTLA-4, for use in the treatment of pancreatic cancer, wherein the treatment is performed according to the method of any one of the previous claims.

69. Gemcitabine, nab-paclitaxel, and an antibody that specifically binds to human CTLA-4, for use in the manufacture of a medicament for the treatment of pancreatic cancer, wherein the treatment is performed according to the method of any one of the previous claims.

70. Use of gemcitabine, nab-paclitaxel, and an antibody that specifically binds to human CTLA-4, for the treatment of pancreatic cancer, wherein the treatment is performed according to the method of any one of the previous claims.

71. A method of treating a subject with cancer that has metastasized to the liver, the method comprising administering to the subject gemcitabine, nab-paclitaxel, and an antibody that specifically binds to CTLA-4.

72. The method of claim 71, wherein the subject has pancreatic cancer.

73. The method of claim 71 or claim 72, wherein the antibody comprises one or more mutations in the Fc region to increase binding to FcγRIIA and/or FcγRIIIA.

74. The method of any one of claims 71-73, wherein the antibody comprises a human IgG1 Fc region comprising S239D/A330L/1332E mutations, numbered according to the EU numbering system.

75. The method of any one of claims 71-74, wherein the antibody that specifically binds to human Cytotoxic T-Lymphocyte Antigen 4 (CTLA-4) is administered at a dose of 25 mg to 250 mg, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

76. The method of any one of claims 71-75, wherein the antibody is administered at a dose of 25 mg to 200 mg.

77. The method of any one of claims 71-76, wherein the antibody is administered at a dose of about 100-200 mg.

78. The method of any one of claims 71-77, wherein the antibody is administered at a dose of about 150-200 mg.

79. The method of any one of claims 71-78, wherein the antibody is administered at a dose of about 150 mg.

80. The method of any one of claims 71-79, wherein gemcitabine is administered at a dose of 600 mg/m², 800 mg/m², or 1000 mg/m².

81. The method of any one of claims 71-80, wherein nab-paclitaxel is administered at a dose of 75 mg/m², 100 mg/m², or 125 mg/m².

82. The method of any one of claims 71-81, wherein the antibody comprises a VH comprising the amino acid sequence set forth in SEQ ID NO: 7 and a VL comprising the amino acid sequence set forth in SEQ ID NO: 8.

83. The method of any one of claims 71-82, wherein the antibody comprises botensilimab.
